(19) United States
(12) Patent Application Publication
(10) Pub. No.: US 2026/0148814 A1
(43) Pub. Date: May 28, 2026

(54) DYNAMIC PATIENT SIMULACRUMS

(71) Applicant: Helix, Inc., San Mateo, CA (US)

(72) Inventors: Jui-Yi Hsieh, San Mateo, CA (US); Magnus Isaksson, Sunnyvale, CA (US)

(21) Appl. No.: 18/990,677

(22) Filed: Dec. 20, 2024

Related U.S. Application Data
(60) Provisional application No. 63/725,432, filed on Nov. 26, 2024.

(51) Int. Cl. G16H 10/60; G16H 50/70

(52) U.S. Cl. CPC G16H 10/60; G16H 50/70

(57) ABSTRACT

Systems and methods are provided for anticipating potential medical conditions for patients. A system includes a controller that retrieves Electronic Health Record (EHR) data for each patient within a population, partitions the EHR data for each patient into discrete time periods, and for each time period for each patient, assembles a sentence comprising a combination of medical concepts for the patient during the time period, resulting in conversion of EHR data for that patient into multiple sentences. The controller also identifies a request to generate a simulacrum for a subject, identifies phenotypes defining the simulacrum, retrieves sentences for a cohort of patients within the population having the identified phenotypes, identifies shared medical concepts between the patients based on the retrieved sentences, and updates the simulacrum to include the shared medical concepts. The controller also generates a command to share the simulacrum in response to the request.

### FIELD

The disclosure relates to the field of health care, and in particular to the selection of patients having shared phenotypes in order to identify clinicogenomic insights.

### BACKGROUND

Medical practitioners desire the ability to not just react to existing medical conditions of a patient, but to provide care to a patient which prevents or delays the occurrence of undesirable medical conditions (e.g., heart attack, stroke, cancer). However, each patient is different, and represents a unique combination of phenotype and clinical history. This means that it is difficult to predict future medical conditions for a patient based on population data, because the population data represents most-likely outcomes across the entire population, and fails to take into account unique trends for sub-populations that the patient may belong to.

Healthcare providers therefore continue to seek out new, robust solutions that enhance the ability to derive personal insights for patients that are both accurate and actionable.

### SUMMARY

Embodiments described herein generate simulacrums that represent potential outcomes for subjects, based upon bespoke cohorts of patients that have similar medical histories to those subjects.

One embodiment is a system for anticipating potential medical conditions for patients. The system includes a controller that retrieves Electronic Health Record (EHR) data for each patient within a population, partitions the EHR data for each patient into discrete time periods, and for each time period for each patient, assembles a sentence comprising a combination of medical concepts for the patient during the time period, resulting in conversion of EHR data for that patient into multiple sentences. The controller also identifies a request to generate a simulacrum for a subject, identifies phenotypes defining the simulacrum, retrieves sentences for a cohort of patients within the population having the identified phenotypes, identifies shared medical concepts between the patients based on the retrieved sentences, and updates the simulacrum to include the shared medical concepts. The controller also generates a command to share the simulacrum in response to the request. The system also includes an interface that transmits the simulacrum toward a source of the request.

A further embodiment is a method for anticipating potential medical conditions for patients. The method includes retrieving Electronic Health Record (EHR) data for each patient within a population, and partitioning the EHR data for each patient into discrete time periods. The method also includes, for each time period for each patient: assembling a sentence comprising a combination of medical concepts for the patient during the time period, resulting in conversion of EHR data for that patient into multiple sentences. The method also includes identifying a request to generate a simulacrum for a subject, identifying phenotypes defining the simulacrum, retrieving sentences for a cohort of patients within the population having the identified phenotypes, identifying shared medical concepts between the patients based on the retrieved sentences, updating the simulacrum to include the shared medical concepts, and transmitting the simulacrum toward a source of the request in response to the request.

A further embodiment is a non-transitory computer readable medium embodying programmed instructions which, when executed by a processor, are operable for performing a method for anticipating medical conditions for patients. The method includes retrieving Electronic Health Record (EHR) data for each patient within a population, and partitioning the EHR data for each patient into discrete time periods. The method also includes, for each time period for each patient: assembling a sentence comprising a combination of medical concepts for the patient during the time period, resulting in conversion of EHR data for that patient into multiple sentences. The method also includes identifying a request to generate a simulacrum for a subject, identifying phenotypes defining the simulacrum, retrieving sentences for a cohort of patients within the population having the identified phenotypes, identifying shared medical concepts between the patients based on the retrieved sentences, updating the simulacrum to include the shared medical concepts, and transmitting the simulacrum toward a source of the request in response to the request.

Other illustrative embodiments (e.g., methods and computer-readable media relating to the foregoing embodiments) may be described below. The features, functions, and advantages that have been discussed can be achieved independently in various embodiments or may be combined in yet other embodiments, further details of which can be seen with reference to the following description and drawings.

### DESCRIPTION OF THE DRAWINGS

Some embodiments of the present disclosure are now described, by way of example only, and with reference to the accompanying drawings. The same reference number represents the same element or the same type of element on all drawings.

FIG. 1 is a diagram depicting a sample processing architecture in an illustrative embodiment.

FIG. 2 is a block diagram illustrating a genomics architecture in an illustrative embodiment.

FIG. 3 is a flowchart depicting a method for automatically generating a simulacrum for a subject to facilitate detection of current and potential future medical conditions for that subject in an illustrative embodiment.

FIG. 4 is a block diagram depicting a simulacrum for a subject in an illustrative embodiment.

FIG. 5 is a block diagram depicting patient data in an illustrative embodiment.

FIG. 6 is a block diagram depicting processing of patient data for multiple patients to generate sentences for a cohort in an illustrative embodiment.

FIG. 7 is a block diagram depicting relations between medical concepts reported within sentences for a cohort in an illustrative embodiment.

FIG. 8 is a flowchart depicting a method for selectively deciding whether to include medical concepts in a simulacrum, based on a prevalence of the medical concepts within a cohort of patients in an illustrative embodiment.

FIG. 9 is a flowchart depicting a method for identifying temporal relationships between medical concepts in a simulacrum, in an illustrative embodiment.

FIG. 10 is a flowchart depicting a method for categorization of medical concepts for selective inclusion within a simulacrum, in an illustrative embodiment.

FIG. 11 is a flowchart depicting a method for selectively deciding whether to include patients within a cohort, based on age and genetic criteria in an illustrative embodiment.

FIG. 12 is a flowchart depicting a method for utilizing a graph data structure to select phenotypes defining a simulacrum, in an illustrative embodiment.

FIG. 13 depicts a graph data structure in an illustrative embodiment.

FIG. 14 depicts a selection of nodes within a graph data structure in an illustrative embodiment.

FIG. 15 depicts processing of natural language content from a request in an illustrative embodiment.

FIG. 16 is a block diagram depicting definitions for simulacra in an illustrative embodiment.

FIG. 17 is a block diagram that depicts summary statistics for a cohort in an illustrative embodiment.

FIG. 18 is a table that summarizes sequencing data for patients and is maintained at a health server in an illustrative embodiment.

FIG. 19 is a table that summarizes variant data for patients and is maintained at a health server in an illustrative embodiment.

FIG. 20 is a table that summarizes biomarker test data for patients and is maintained at a health server in an illustrative embodiment.

FIGS. 21-22 depict Graphical User Interfaces (GUIs) that facilitate the creation and review of simulacrums for patients in illustrative embodiments.

FIG. 23 depicts an illustrative computing system operable to execute programmed instructions embodied on a computer readable medium.

### DESCRIPTION

The figures and the following description depict specific illustrative embodiments of the disclosure. It will thus be appreciated that those skilled in the art will be able to devise various arrangements that, although not explicitly described or shown herein, embody the principles of the disclosure and are included within the scope of the disclosure. Furthermore, any examples described herein are intended to aid in understanding the principles of the disclosure, and are to be construed as being without limitation to such specifically recited examples and conditions. As a result, the disclosure is not limited to the specific embodiments or examples described below, but by the claims and their equivalents.

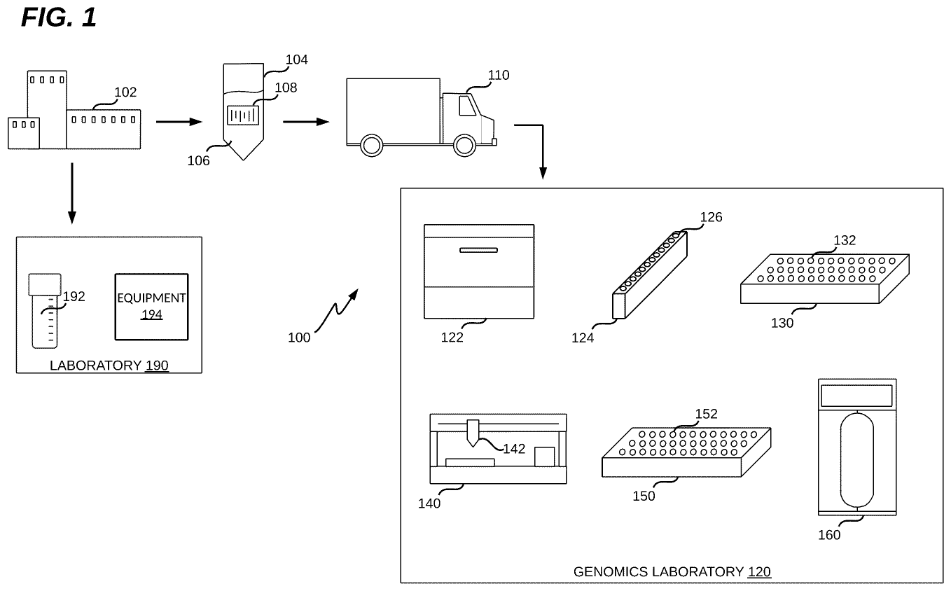

FIG. 1 is a diagram depicting a sample processing architecture 100 in an illustrative embodiment. Sample processing architecture 100 comprises any system or organizational structure for acquiring and sequencing biological samples in a high-volume, high-throughput manner. Sample processing architecture 100 may be utilized, for example, to collect and sequence genetic material (in the form of Ribonucleic Acid (RNA) or Deoxyribonucleic Acid (DNA)) found within thousands or tens of thousands of samples 106 daily, via multiple healthcare provider networks 102.

Healthcare provider networks 102 may comprise hospitals, clinics, practitioner offices, laboratories, surgical centers, etc. that engage in or facilitate the practice of medicine. In one embodiment, healthcare provider networks 102 each comprise groups of hospitals that treat millions of patients. As a part of the practice of medicine, healthcare provider networks 102 acquire samples 106 for sequencing. For example, a healthcare provider network 102 may acquire samples 106 as part of a population screening program, as part of medical treatment, etc. The specific amount of sequencing desired for a sample 106 may comprise a selected set of one or more genes, an exome, the entire genome of a patient, etc. The samples 106 are stored in sample containers 104, which may be accompanied by Customer Sample Identifiers (CSIs) 108. A delivery service 110 provides the samples 106 to a genomics laboratory 120 for processing.

Healthcare provider networks 102 may also acquire samples 192 for conventional blood testing (described below). These samples 192 may be provided to laboratory 190 for analysis via equipment 194 (e.g., a chemically treated test strip, biochemical assay, etc.), or may be analyzed by patients via at-home testing methods. Sample processing architecture 100 provides a technical benefit by allowing laboratory 190 and genomics laboratory 120 to specialize in different methods of analysis.

Procedures within genomics laboratory 120 related to genetics may include accessioning, sample plating, storage, extraction, library preparation, enrichment, and sequencing processes. These processes acquire genetic material from a sample 106, separate the genetic material from other constituents, duplicate the genetic material, and quantify the genetic material order to determine a swathe of sequence data, such as an exome or entire genome for a subject (e.g., a human patient, an organelle of a human patient, etc.). Although the procedures discussed herein are specific with regard to one method of sequencing, other techniques may be utilized in accordance with known standards in order to perform sequencing for samples 106. For example, although certain short-read technologies herein are discussed as utilizing hybridization capture techniques, amplicon-based techniques may be used alternatively or to supplement those techniques. Long-read techniques may also or alternatively be utilized.

### Accessioning

Accessioning refers to receiving and preparing samples 106 for later laboratory processes. In one embodiment, accessioning includes receiving a batch of samples 106 (e.g., hundreds or thousands of samples 106) from one or more delivery services 110 each day for processing. For example, packages that each include tens or hundreds of samples 106 may be delivered to genomics laboratory 120 via the United States Postal Service (USPS), or a private package carrier.

Each sample 106 may be retained within a sample container 104, such as a five milliliter (mL) test tube. In this embodiment, the sample container 104 is sealed to prevent the sample 106 from being exposed to the environment and also to prevent the sample 106 from co-mingling with other samples 106. For example, the sample 106 may be sealed via a cap that is threaded, glued, press-fit, etc. At the time of delivery, the sample container 104 may further include a remnant of a sampling tool, such as a portion of a swab that was utilized to acquire the sample.

In many embodiments, a CSI 108 for the sample 106 is reported via a component affixed to or integrated with the sample container 104. The CSI 108 uniquely distinguishes the sample 106 from other samples 106 being received. For example, a CSI 108 may uniquely distinguish a sample 106 from other samples 106 in the same batch, other samples 106 received on the same date, other samples 106 received from the same healthcare provider network 102, etc. A CSI 108 may be reported via a barcode label, Quick Response (QR) code label, Radio Frequency Identifier (RFID) chip, or any suitable visual, transmission-generating, or other physical component affixed to or integrated with the sample container 104.

In further embodiments, the sample container 104 is itself sealed within an external container such as a bag (not shown). Using an external container helps to prevent contamination, by ensuring that a technician at the genomics laboratory 120 does not contact biological material from the sample 106 that may exist on an outer surface of the sample container 104. Use of an external container may also be required by law (e.g., Department of Transportation (DOT) guidelines). Use of an external container additionally helps to prevent cross-contamination between samples 106. Furthermore, in embodiments where samples 106 may include blood or a pathogen, an external container provides an additional barrier to protect the health of technicians. The external container may additionally include documentation confirming the CSI 108, information for the subject that the sample was sourced from, and/or information indicating circumstances of sampling. The circumstances of sampling may include, for example, a sampling date, a sampling method, a location that the sample was acquired, a name or title for a person who performed the sampling, and/or additional notes.

In this embodiment, the sample 106 comprises a chemical solution. For example, the sample 106 may comprise a prepared aqueous solution such as a saline solution, or may comprise a bodily fluid such as whole blood, blood spots, saliva, buccal material, mucus, etc. In some embodiments each of the samples 106 fills between two and five milliliters of volume within its corresponding sample container 104.

The samples 106 further include genetic material such as Deoxyribonucleic Acid (DNA), Ribonucleic Acid (RNA), etc. In many instances, the genetic material is one of many constituent components within the sample 106. For example, the genetic material may exist within the nuclei of white blood cells that are included within the sample 106. In a further example, genetic material may exist within viruses or bacteria within the sample 106. In this embodiment, the genetic material is not yet isolated from the remaining constituent components of the sample 106.

After receipt of the samples 106, batches of the samples 106 (e.g., as stored within sample containers 104 and/or external containers) may be heated in ovens 122 to facilitate cell lysis. The temperature, and duration of heating, may be chosen such that pathogenic material within the samples 106 is rendered harmless, or such that cellular lysis occurs. For example, heating may occur at a temperature of between forty and eighty (e.g., fifty) degrees Celsius (C), for a period of time between fifteen and two hundred (e.g., thirty) minutes. In some embodiments, including embodiments wherein the samples 106 are primarily blood or buccal material, the heating step may be foregone.

In this embodiment, upon completion of heating, the batches of samples 106 are removed from the ovens 122. In one embodiment, sample containers 104 are removed from corresponding external containers, such as by cutting the external containers open. With the sample containers 104 now available for direct interaction, the sample containers 104 are inspected. As a part of this process, a technician or automated system may determine the CSI 108 for the sample 106, and may compare the CSI 108 to a CSI 108 listed on documentation provided in the external container. If there is a discrepancy between the CSI 108 on the sample container 104 and a CSI 108 listed in the documentation, the sample 106 may be flagged as having an error condition. Similarly, if the CSI 108 on the sample container 104 is damaged (e.g., abraded, heat-damaged, or water-damaged) and has become unreadable, the sample 106 may be flagged as having an error condition.

A technician or automated system may further inspect the contents of the sample container 104, via visual or other methods. If the sample 106 does not include an expected constituent component (or is otherwise non-compliant) then the sample 106 is flagged as having an error condition. For example, if the sample 106 is primarily saliva and includes a fluid that is not permitted (e.g., blood), includes an entire swab or no swab, appears to have a fractured or broken casing, or is outside of an expected range of volume (e.g., between two and five milliliters), then the sample 106 may be flagged as having an error condition.

Samples 106 that have not been flagged as having an error condition proceed to sample integration. In one embodiment, as a part of sample integration, the sample 106 is assigned a Laboratory Sample Identifier (LSI). The LSI uniquely identifies the sample 106 from other samples 106 received for the batch, received on the same day, processed in the same laboratory, and/or handled by the same organization performing sequencing. In many embodiments, the LSI is stored in a memory of a health server (e.g., within a laboratory sample database), and is uniquely associated with a corresponding CSI 108 for the sample. The LSI may also be associated with any error conditions reported for the sample 106.

In many embodiments, CSIs 108 originally provided with the samples 106 are in the form of a paper barcode. In such embodiments, the paper barcode may be printed in aqueous ink. This renders the barcode subject to degradation upon exposure to liquid in the laboratory environment, which is undesirable.

To ensure that each sample container 104 is capable of traveling through the genomics laboratory 120 without its identifier being physically degraded, a corresponding LSI may be indicated at the sample container 104. The LSI may be indicated via the application of a barcode label, Quick Response (QR) code, Radio Frequency Identifier (RFID) chip, or other visual, transmission-generating, or other physical component affixed to or integrated with the sample container.

In one embodiment, the LSI is printed onto a barcode label comprising rip-proof material (e.g., vinyl) in a water-insoluble ink. This implementation ensures that the barcode label is resistant to physical and chemical degradation. The barcode may be applied around an entire perimeter of the sample container 104, ensuring that the sample container 104 may be scanned from any angle.

In further embodiments, the element used to report the LSI is accompanied by a visually distinct mark that enables rapid confirmation by a technician that the sample 106 has been integrated into the laboratory environment. The visually distinct mark may comprise a colored ring (e.g., around an entire perimeter of the sample container), a logo, a physical feature, a stamp, etc.

### Sample Plating

With the samples 106 having been successfully integrated into the environment of the genomics laboratory 120 environment, the samples 106 are ready for analytics to be performed. To this end, the samples 106 are prepared for transfer to a sample microplate 130. The sample microplate 130 may be labeled with a unique identifier via similar techniques to those used for sample containers 104 above. The unique identifier distinguishes the sample microplate 130 from other sample microplates 130. In one embodiment, the sample microplate 130 comprises a solid body defining three hundred and eighty-four wells, distributed across sixteen rows and twenty-four columns, each well having a capacity of between thirty and one hundred microliters. In a further embodiment, the sample microplate 130 comprises a solid body defining ninety-six wells, distributed across eight rows and twelve columns, each well having a capacity of between one hundred and three hundred microliters. Any suitable number and arrangement of wells may be selected as a matter of design choice.

As a part of preparing the samples 106 for transfer to the sample microplate 130, a technician may place sample containers 104 onto a rack 124, and scan each sample container 104 to determine an LSI for each location 126 (e.g., each container receptacle) on the rack 124. In some embodiments, the rack 124 is assigned a unique identifier that distinguishes it from other racks 124. The rack 124 may be labeled with a unique identifier using techniques similar to those used for sample containers 104. The technician, or automated machinery such as a server operating an optical scanner, may then associate the unique identifier for the rack 124, along with the locations 126 assigned to the samples 106, with the corresponding LSIs of the samples 106 stored at the rack 124.

The technician additionally unseals the sample containers 104. Unsealing of sample containers 104 may be a deeply labor-intensive process, particularly when laboratory processes are performed at scale to handle tens of thousands of samples 106 per day. Thus, a technician may utilize automated tooling to enhance the speed at which sample containers 104 are unsealed. The tooling may, for example, unscrew, cut, or drill each sample container 104, in order to make the sample 106 within available for physical transfer to the sample microplate 130.

One or more racks 124 of samples 106 are provided to a Liquid Handler (LH) 140, such as an automated robot that operates an end effector 142 in accordance with one or more Numerical Control (NC) programs to transfer liquids between wells via arrays of micropipettes. An LH 140 is also known as a “Liquid Handling System.” LH 140 may comprise, for example, a Hamilton Microlab Star Liquid Handling System.

In this embodiment, the LH 140 proceeds to transfer a portion of each sample 106 at a rack 124 to a well 132 within the sample microplate 130 that is not shared with other samples 106. For example, the well 132 for each sample 106 may be predetermined in accordance with a control program used by the genomics laboratory 120. In one embodiment, the LH 140 transfers the portions of the samples 106 to the wells 132 of the sample microplate 130 by providing instructions to actuators, piezoelectric elements, and/or pressure systems operating the end effector 142. In such an embodiment, the end effector 142 may align its array of micropipettes with the sample containers 104 to retrieve portions of the samples 106. Furthermore, in such an embodiment, the end effector 142 may dynamically align its array of micropipettes with the sample microplate 130 to deposit the portions of the samples 106 at the wells 132.

Because there is a known relationship between locations 126 at the rack 124 and wells 132 of the sample microplate 130 (e.g., as indicated by row and column), contents of the memory of a health server (e.g., a laboratory sample database) may be updated to indicate the well 132 storing genetic material for each sample 106. In one embodiment, the memory is further updated to associate a unique identifier for the sample microplate 130 with the samples 106 stored therein.

In one embodiment, programmed instructions for the LH 140 may direct the end effector 142 to position itself above a set of disposable tips, descend into the tips to attach the tips, reposition the end effector 142 above the rack of sample containers 104, adjust spacing between micropipettes within the array, descend until the tips reach the sample containers 104, draw liquid from the sample containers 104, deposit the liquid into a well at the sample microplate 130, and then dispose of the tips. Such a process may be repeated across sample containers 104 stored on multiple racks until the sample microplate 130 is filled with portions from the samples 106. In one embodiment, one or more wells 132 on the sample microplate 130 are filled with a control reagent instead of a portion of a sample 106.

The amount of liquid drawn from each sample container 104 may comprise a small fraction of the overall volume of the sample container 104. For example, an amount of liquid drawn may comprise several microliters, such as between two and ten microliters. Upon completion of transfer from the sample containers 104 to the wells, the sample microplate 130 may be covered with a liquid and/or gas-impermeable layer, such as foil or paraffin. Sample containers 104 remaining on the racks may be resealed, for example with pressure-fit caps having a color distinct from an original color for the sample containers. With accessioning now complete for the sample microplate 130, the sample microplate 130 is transferred to a next section of the laboratory for processing.

In embodiments wherein the genomics laboratory 120 performs both short-read and long-read sequencing workflows, the sample plating techniques discussed above may be performed separately, asynchronously, and/or in parallel for short-read technologies (e.g., via an Illumina sequencing platform such as a NovaSeq X) and for long-read technologies (e.g., via a PacBio sequencing platform such as a Revio). Samples 106 received at the genomics laboratory 120 may include sufficient genetic material to support multiple sequencing processes (e.g., both short-read and long-read sequencing processes).

### Storage

In one embodiment, accessioned samples 106, samples 106 ready for analytics, and/or samples 106 that have already been sequenced, are stored for later use. For example, samples 106, sample containers 104, and/or sample microplates 130 may be stored at room temperature, or may be cryogenically frozen at a low temperature (e.g., negative eighty degrees Celsius) and arranged in racks for later retrieval. Samples 106 may be preserved for periods of days or years, enabling rapid re-testing to be performed for subjects without the need for re-acquiring genetic material. Storage of the samples 106 provides notable value in the event that contents of a well 132 used for sequencing do not meet with rigorous quality control standards. Specifically, storage enables re-sampling to occur in the event that there is a desire to re-sequence a sample 106.

### Extraction

Sample microplates 130 are transferred to a portion of the genomics laboratory 120 dedicated to extraction of the genetic material. The segment of the laboratory 120 that performs extraction and other pre-amplification operations may be sealed from, and/or positively pressurized relative to, other portions of the genomics laboratory 120.

During extraction, a sample microplate 130 is acquired and provided to an LH 140. The LH 140 that performs extraction may be different from the LH 140 that performs sample plating. The LH 140 may apply a reagent to each well 132 that lyses cells within each well. For example, this may be performed in order to lyse white blood cells containing genetic material for a human, or may comprise lysing other types of cells to expose other types of genetic material. The reagents used for pre-amplification processes may be stored at the LH 140 in a temperature-controlled manner, and may even be vibrated or mixed on a regular basis to ensure that the reagents are evenly distributed in suspension.

In one embodiment, extraction further includes an LH 140 aspirating and dispensing reagents that selectively bind to genetic material released from the lysed cells. This process may include applying a bead (not shown) to the well 132. In one embodiment, the beads comprise magnetic beads that selectively bind to the genetic material (e.g., DNA). This allows for isolation and purification of the genetic material while contaminants remain in solution. In one embodiment, the magnetic bead is drawn to a magnetic base at or under the sample microplate 130. After the genetic material has been drawn to the bead, and after the bead has been secured to the base of the well, a flushing step may be performed wherein remaining fluid in each well is washed away. This ensures that potential impurities are removed from the well. The LH 140 may further add or remove fluid from each well 132 to perform additional concentration and/or elution of the genetic material, and may transfer fluid from the wells 132 of the sample microplate 130 to wells 152 of a genome stock microplate 150. The genome stock microplate 150 may be labeled with a unique identifier, and the contents of each well 152 of the genome stock microplate 150 may be associated with a corresponding LSI. In all phases of operation, the LH 140 is operated to ensure that fluid is not transferred between wells 152, as this results in contamination.

In one embodiment, a portion of fluid is removed from each well 152 of the genome stock microplate 150 for quality control purposes. Concentration of genetic material within the wells 152 may be confirmed via testing of this fluid, such as by application of a dye that reacts with the genetic material at known levels of fluorescence for known concentrations.

In embodiments where the genomics laboratory 120 performs both short-read and long-read sequencing workflows, the extraction techniques discussed above may be performed separately, asynchronously, and/or in parallel for short-read technologies (e.g., via an Illumina sequencing platform such as a NovaSeq X) and for long-read technologies (e.g., via a PacBio sequencing platform such as a Revio).

### Library Preparation

After extraction is completed, library preparation may be performed for the contents of the genome stock microplate 150. The bead for each well, including ionically bonded genetic material, is transferred to a distinct well of a library preparation microplate (not shown). The library preparation microplate includes an identifier that uniquely distinguishes it from other library preparation microplates, and the LSI associated with each well on the genome stock microplate 150 may be mapped to a corresponding well on the library preparation microplate.

The library preparation microplate may be transferred to a new portion of the genomics laboratory 120 that is sealed from, and/or positively pressurized relative to, other portions of the genomics laboratory 120 that do not perform amplification of genetic material. This feature helps to prevent amplified genetic material from entering portions of the laboratory where genetic material has not been amplified, which could result in contamination. The transfer process may be performed by placing a library preparation microplate into an airlock at the pre-amplification portion of the genomics laboratory 120, sealing the airlock, and then retrieving the library preparation microplate from the airlock via the amplification portion of the genomics laboratory 120.

In one embodiment, a reagent is applied to each well of the library preparation microplate. The reagent ionically bonds to the surface of the bead within the well, and does so more strongly than the genetic material. This releases the genetic material from the surface of the bead of each well, enabling the genetic material to be chemically interacted with.

Library preparation may include normalization of a concentration of genetic material in each well of the library preparation microplate. Library preparation further includes fragmentation of the genetic material via an enzyme or via the application of physical forces. During this process, the entire genome (e.g., roughly three billion base pairs for a human genome), may be fragmented into pieces. In one embodiment where short-read sequencing is performed, the pieces vary between three hundred and four hundred base pairs in length. These pieces are known as nucleic acid fragments. In a further embodiment where long-read sequencing is performed, the pieces may vary between five hundred and fifty thousand or more base pairs in length.

In one embodiment utilizing short-read sequencing, the nucleic acid fragments undergo adaptor ligation and indexing in accordance with known techniques. For example, this may comprise Next Generation Sequencing (NGS) library preparation processes defined by Illumina. Next, a limited amount of Polymerase Chain Reaction (PCR) amplification is performed upon the library. The resulting solution is then purified and eluted via operation of an LH 140.

During library preparation, one or more reference samples of genetic material, distinct from the genetic material found in the samples, may be added to wells of the library preparation microplate. The reference samples do not include genetic material received from a customer, but rather include known sequences of base pairs. The reference samples serve as controls to ensure that processes are carried out with sufficient quality.

Upon completion of library preparation, desired fragments of the genetic material (e.g., thousands or millions of distinct fragments of the genetic material, each corresponding with a different portion of a genome of the subject) have been ligated to predefined adapters (e.g., DNA adapters) that bind with the genetic material. Each of the adaptor-ligated fragments is referred to as a “library.”

In further embodiments, the probes applied to each well of the library preparation plate include chemical identifiers (colloquially referred to as “barcodes”) that are distinct from each other. The use of a different chemical identifier for probes applied to each well of the library preparation microplate enables sequencing to later be performed for multiple subjects on the same flow cell, without conflating sequencing results for those subjects.

In one embodiment utilizing long-read sequencing, library preparation may be performed via physical shearing of DNA to achieve a target size distribution mode between ten and twenty-five kilobases (kb), such as between fifteen and eighteen kb. The resulting nucleic acid fragments may be coupled to adapters to prepare them for sequencing via Single-Molecule Sequencing in Real Time (SMRT) or other long-read technologies.

The library preparation processes discussed herein may further comprise controlling a concentration of the genetic material in each well, and purification and/or elution of the resulting material. Similar to the processes performed after extraction of genetic material, concentration of genetic material after library preparation may be confirmed for each well via testing.

### Enrichment

After library preparation, enrichment processes may be performed in order to either directly amplify (e.g., via amplicon or multiplexed PCR) or capture (e.g., via hybrid capture) predefined libraries. This enhances the ease of sequencing desired portions of the genome. In some embodiments, enrichment is foregone for long-read sequencing processes.

In one embodiment, during enrichment, customized biotinylated oligonucleotide probes are applied to the libraries. The probes selectively hybridize genetic material occupying desired portions of the genome for the genetic material, such as specific genes, or the entire exome. Magnetic beads bind to biotin molecules in the probes to attach the hybridized material to the magnetic beads. Magnetic forces capture the beads in place, enabling remaining fluid within each well to be removed or washed out, thereby removing impurities and leaving only the genetic material that is desired. Genetic material may be released from the beads in a similar manner to that discussed above for prior processes.

In a further embodiment, hybrid capture target enrichment is performed. During this process, the probes comprise tailored oligonucleotides that are chosen to bind to the genetic material. The range of probes may be tailored as a group to bind to specific alleles, specific genes, the exome, the entire genome, etc. That is, each probe may bind to a nucleic acid fragment at a specific location on the genome, and the range of probes may be selected to ensure that alleles, genes, the exome, or the entire genome of the subject being considered is acquired. Utilizing probes in this manner may enhance efficiency of the sequencing process, by foregoing sequencing of undesired portions of the roughly three billion base pairs found in the human genome.

The enrichment process may further comprise controlling a concentration of the genetic material in each well, and purification and/or elution of the resulting material. Similar to the processes performed after extraction of genetic material, concentration of genetic material after enrichment may be confirmed for each well via testing.

### Sequencing

Sequencing may be performed according to any of a variety of techniques, including short-read and long-read techniques, via sequencing equipment 160 (e.g., an Illumina NovaSeq X sequencing machine, a PacBio Revio sequencing machine, etc.). As used herein, short-read sequencing refers to sequencing technologies that generate reads of less than five hundred base pairs in length. Short-read sequencing may be used as the basis for “synthetic long read” technologies that stitch individual short reads together, but as used herein, short-read sequencing does not refer to the creation or use of synthetic long reads.

In one embodiment, short-read sequencing is performed as Sequencing by Synthesis (SBS). For example, sets of enriched libraries of genetic material bound to probes in earlier steps may be transferred to a flow cell, and annealed to oligonucleotide probes within the flow cell. At this stage, the contents of multiple wells may be applied to the same flow cell, because the libraries within those wells are tagged with the chemical identifiers referred to above. In one embodiment, the chemical identifiers comprise nucleotide sequences that are detectable during the sequencing process to determine a corresponding LSI.

Complementary sequences may then be created via enzymatic extension to create a double-stranded portion of genetic material. The double-stranded genetic material may then be denatured, and the library fragment may be washed away. Bridge amplification may then be performed to create copies of the remaining molecule in a localized cluster. For example, a cluster may comprise twenty to fifty copies of the same molecule, localized to a location the size smaller than a pinhead on the flow cell.

In this embodiment, sequencing primers are annealed to library adapters in order to prepare the flow cell for SBS. During SBS, the sequencing primer uses reverse terminator fluorescent oligonucleotides, one base per cycle, for a number of cycles (e.g., one hundred and fifty cycles) in the forward direction. After the addition of each nucleotide, clusters are excited by a light source, resulting in fluorescence which can be measured. The emission wavelength and signal intensity for each cluster determines a base call for that cluster. Fluorescent moieties are then flushed from the flow cell. A chemical group blocking a 3′ end of the fragment is then removed, enabling a subsequent nucleotide to be read. This tightly controls nucleotide addition and detection.

Additionally in this embodiment, base calls across cycles at the same physical location on the flow cell occur at the same cluster, and hence indicate sequential reads for copies of the same fragment of the genetic material. After each cycle, denaturing and annealing are performed to extend the index primer. A complementary reverse strand is created and extended via bridge amplification. The reverse strand is then read in the reverse direction for a number of cycles, in a manner similar to reads in the forward direction.

Depending on whether a complete human genome, or another set of genomic data, is being tested, different reagents (e.g., probes, primers, etc.) may be chosen. That is, different reagents may be utilized for library preparation for a pathogen (e.g., bacteria, virus) or an organelle (e.g., mitochondria) than for a human genome. Pathogens exhibiting Ribonucleic Acid (RNA) genomes may have their genetic material translated to DNA before sequencing, enrichment, and/or library preparation are performed, via known techniques, such as Next Generation Sequencing (NGS) techniques.

In a further embodiment, long-read sequencing (e.g., sequencing of nucleic acid fragments larger than one kilobase) is performed in a Single-Molecule Sequencing in Real Time (SMRT) process, wherein nucleic acid fragments are circularized and bound to a DNA polymerase enzyme. The bound pair enter a sequencing chamber, and the DNA polymerase adds complementary bases to the DNA strand that are fluorescently labeled to result in different colors for different bases.

As labelled bases are added by the polymerase, the color of the base is recorded, and then the fluorescent label is removed. The next base for the circularized nucleic acid fragment is then added and recorded, iteratively, until the circularized nucleic acid fragment has been sequenced a desired number of times.

Throughout the processes discussed above, the laboratory environment may be carefully controlled to ensure quality. For example, temperature within each segment of the laboratory may be carefully monitored and controlled, and ultraviolet lighting or other features capable of inactivating genetic material may be carefully positioned to ensure that contamination does not occur.

### Bioinformatics

Sequencing data may be stored in any suitable format. In one embodiment, raw sequencing data generated during short-read sequencing is stored in a file format such as Binary Base Call (BCL). This raw data may be fed to an analytical pipeline such as a cloud-based computing environment. Raw sequencing data may be processed by the pipeline into a second format, such as a text-based FASTQ format, that reports quality scores. The second format may then be analyzed to perform alignment of sequence reads to a reference genome, such as a reference genome reported in a Browser Extensible Data (BED) file. The aligned sequence data may be reported as a Binary Alignment Map (BAM) file or Compressed Reference-oriented Alignment Map (CRAM) file. In one embodiment, long-read sequencing data is output from the corresponding sequencing machine as one or more BAM files, obviating the need for long-read sequence data undergoing the conversion processes discussed above.

The aligned sequence data may then be called, resulting in a Variant Call Format (VCF) file reporting called variants at each location of the genome that was sequenced, together with secondary metrics such as quality indicator metrics. As used herein, a variant comprises a unique combination of genetic information, such as in the form of consecutive base pairs at a specific set of locations (e.g., genomic coordinates) along a portion of a chromosome. Each variant is distinguished from other variants by having a different combination of base pairs along the set of locations. This may be due to Single Nucleotide Polymorphisms (SNPs) which relate to common single nucleotide changes, Single Nucleotide Variants (SNVs) which relate to rare nucleotide changes, insertions and/or deletions (Indels) which relate for example to the insertion or deletion of less than thirty base pairs, or differing numbers of repetitions, Copy Number Variants (CNVs), which relate to larger insertions or deletions, translocations, inversions, other types of genetic variants, or even combinations of variants, such as haplotypes or Multi-nucleotide variants (MNVs).

The called sequence data may be provided to a data analyst via a User Interface (UI), such as a Graphical User Interface (GUI) presented via a display. The technician may then validate the resulting called sequence data and release it for reporting to subjects, health care providers, and/or scientists.

### Health Reporting Architecture

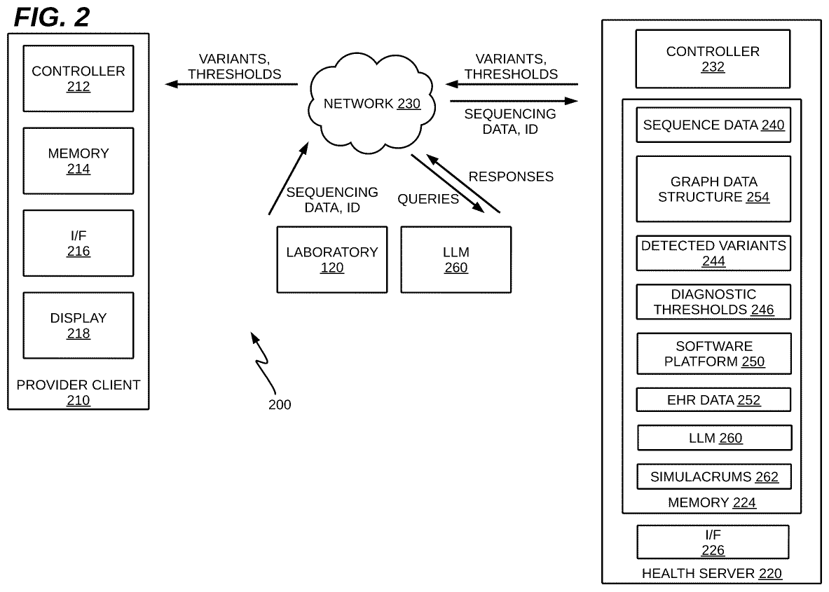

FIG. 2 is a block diagram illustrating a health reporting architecture 200 in an illustrative embodiment. Health reporting architecture 200 comprises any combination of systems and devices operable to review, process, and/or control access to health data, such as Electronic Health Record (EHR) data 252 from healthcare providers, and/or sequencing data received from genomics laboratory 120. In this embodiment, health reporting architecture 200 comprises a health server 220 which receives sequencing data and identifiers (e.g., CSIs 108, LSIs, etc.) from genomics laboratory 120, via network 230. The sequencing data received and processed by the health server 220 may be supplied for multiple different types of sequencing operations, including short-read and long-read sequencing operations.

Health server 220 receives the sequencing data via interface (I/F) 226, such as an Ethernet interface, wireless interface compliant with Institute of Electrical and Electronics Engineers (IEEE) 802.11 standards, or other physical interface capable of transmitting and receiving digital data. The sequencing data 240 is stored in memory 224 for the population of patients (e.g., millions of patients) that have been sequenced by laboratory 120, and may be maintained in any suitable format. Examples of such formats include CRAM, VCF, BAM, and others. Memory 224 may store, for example, sequence data 240 describing multiple patients, and this sequence data 240 may be maintained in a de-identified format to facilitate the advancement of research. Memory 224 may be implemented via a cloud storage service, or may comprise a storage medium such as a hard disk or flash memory device.

Memory 224 may additionally store detected variants 244 found for individual patients, and diagnostic thresholds 246 for diagnosis and/or treatment of specific diseases. In one embodiment, the portion of memory 224 storing these components is distinct from the portion of memory 224 storing sequence data 240.

Memory 224 further stores software platform 250 for directing interactions between users and a tool for generating simulacrums of patients. In one embodiment, the code for software platform 250 is maintained as code in javascript, Hypertext Markup Language (HTML) five, or other browser-friendly formats.

In some embodiments, the software platform 250 consults a Large Language Model (LLM) 260, which may be integrated into software platform 250. Additionally, in some embodiments, software platform 250 calls upon one or more LLMs 260 hosted by third parties available via network 230 (i.e., as depicted in FIG. 2). In particular, software platform 250 facilitates the building simulacrums 262 that each represent, on a bespoke, per-patient basis, evidence-supported potential outcomes and future medical concepts or conditions for a specific patient.

In one embodiment, software platform utilizes graph data structure 254 to facilitate the building of simulacrums 262. Graph data structure 254 comprises a graph that stores knowledge as nodes and edges/relationships, rather than via a relational database comprising structured tables with rows and columns. Graph data structure 254 includes nodes for multiple medical concepts, and aggregates content from one or more medical vocabularies (e.g., Systematized Nomenclature of Medicine Clinical Terms (SNOMED), Logical Observation Identifiers Names and Codes (LOINC), RxNorm, International Classification of Diseases (ICD) Clinical Modification, such as ICD10-CM, Current Procedural Terminology (CPT), Observational Medical Outcomes Partnership (OMOP) Common Data Model (CDM) vocabularies, and/or others), to facilitate rapid identification of related concepts. In one embodiment, graph data structure 254 has been refined to add edges/relationships representing inter-vocabulary relationships, such as those between diagnosing a medical concept (e.g., diabetes) in an ICD vocabulary, and treating for the medical concept in a CPT vocabulary.

As used herein, a “medical concept” comprises a diagnosis, medical procedure, measurement (e.g., lab, vital), medication use, or exposure to a medical device. Hence, medical concepts may encompass diseases, phenotypes, treatments, and/or conditions relating to medical care and treatment, such as those described in standard ontology systems. Examples of phenotypes for medical concepts may include “type II diabetes,” “obese,” or “skin cancer.” Medical concepts are distinguished from medical conditions in that medical conditions tend to be more narrow, refer to specific diseases, disease states, organ functions (such as bradycardia), and/or other states of being directly experienced by patients.

In a further embodiment, memory 224 additionally stores Electronic Health Record (EHR) data 252 for one or more patients. The EHR data 252 may comprise records that have been rendered into a uniform format, such as an OMOP format, and may comprise health records for each patient that sequencing data has been stored for. In one embodiment, the EHR data 252 includes content coded according to one or more medical vocabularies.

Controller 232 manages the operations of health server 220, and may for example analyze sequence data 240 to determine alignments to a reference genome, identify detected variants 244, control access and authentication related to sequence data 240, communicate with one or more provider clients 210, and/or perform additional operations.

Controller 232 may be implemented, for example, as custom circuitry, as a hardware processor executing programmed instructions, as a combination of shared hardware processing resources implementing a compute service, or some combination thereof.

Health reporting architecture 200 further comprises provider client 210, which is configured to permit users to interact with LLM 260 in order to generate cohorts. In some embodiments, provider client 210 is further configured to facilitate genomics-related activities, and receive information regarding detected variants 244 and/or diagnostic thresholds 246.

In the embodiment depicted in FIG. 2, provider client 210 includes a controller 212, a memory 214, an interface (I/F) 216, and a display 218. Controller 212 manages the operations of the provider client 210, and may be implemented, for example, as custom circuitry, as a hardware processor executing programmed instructions, or some combination thereof. Memory 214 comprises information for interpreting the data received via I/F 216. Display 218 may comprise a projector, screen, etc. for presenting information to a user of provider client 210.

After sequencing data for a patient has been acquired, it is maintained at health server 220 in order to facilitate future studies associating relationships between genetic variants and phenotypes. This means that health server 220 has readily available access to clinic-genomic data sets that may be highly desirable for studies of cohorts of patients.

In further embodiments wherein simulacrums generated for patients are not related to genomics, the processes discussed above related to sequencing and storage of sequencing data may be foregone.

### Patient Simulacrum Construction

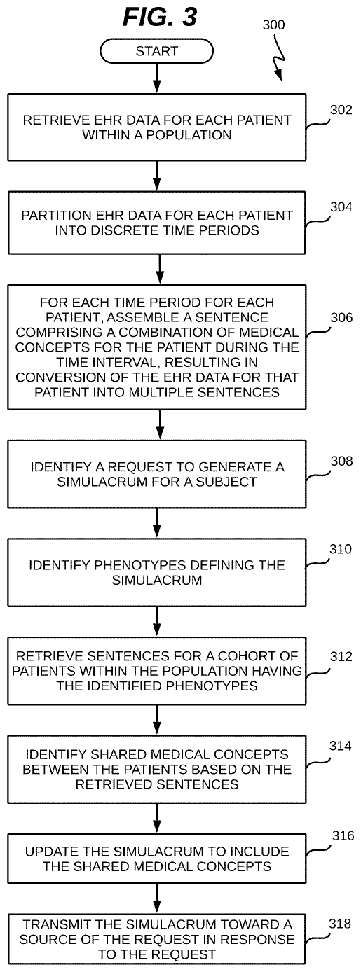

FIG. 3 is a flowchart depicting a method 300 for automatically generating a simulacrum for a subject to facilitate detection of current and potential future medical conditions for that subject in an illustrative embodiment. The steps of the flow charts described herein are not all inclusive and may include other steps not shown, and the steps may be performed in an alternative order. For example, steps that are depicted with dashed lines are explicitly provided as optional in both their inclusion and order, although this may apply to other steps as well. As used herein within the context of this application, a “subject” generally refers to a specific patient or potential patient, rather than a topic or field. By generating a simulacrum based on specific medical concepts found within the subject, health server 220 beneficially derives insights that are specific to that patient, rather than applicable to the general population. Method 300 may be implemented, for example, in collaboration with sample processing architecture 100, health reporting architecture 200, and/or other systems and architectures. For example, although method 300 is described with respect to sequencing processes and methods in this embodiment, such methods are not necessary for method 300 to operate.

Step 302 of method 300 comprises controller 232 retrieving EHR data 252 for each patient within a population, such as a population of patients within one or more healthcare provider networks 102. Retrieving the EHR data 252 may comprise retrieving “raw” EHR records for individual patients, and reformatting those records into a standardized format for storage in memory 224, such as an Observational Medical Outcomes Partnership (OMOP) Common Data Model (CDM) or other format. The EHR data 252 may be received periodically, such as once per quarter, or in real-time as EHR data 252 for patients is updated. In many embodiments, the population comprises hundreds of thousands or even millions of patients, and hence the scale of corresponding EHR data is not feasible to manually process and aggregate.

Step 304 comprises controller 232 partitioning EHR data 252 for each patient in the population into discrete time periods. In one embodiment, step 304 is performed by selecting a uniform interval length for the time periods, such as a week, month, quarter, or year, and then subdividing EHR data 252 such that input added to the EHR data 252 during the same time period are grouped together. In one embodiment, the time periods for a patient are adjacent and contiguous, and the interval length is the same for all time periods and patients.

In further embodiments, step 304 is performed according to methods and techniques discussed within De Freitas et al., “Phe2vec: Automated disease phenotyping based on unsupervised embeddings from electronic health records”, 2021, Patterns 2, 100337, Sep. 10, 2021, the contents of which are herein incorporated by reference.

In step 306, for each time period for each patient, controller 232 assembles a sentence comprising a combination of medical concepts for the patient during the time period, resulting in conversion of EHR data 252 for that patient into multiple sentences. In one embodiment, controller 232 assembles a sentence for a patient by categorizing each piece of data within the corresponding time period for the patient, and assigning a category to each piece of data. Example categories include vitals, lab results/tests, medications, procedures, diagnoses, medical procedures, etc. Controller 232 further assigns each sentence metadata, including an identifier for the corresponding patient, as well as timing information when the time period for the sentence occurred. This facilitates arrangement of the sentences for each patient in order. In a further embodiment, techniques such as those used in De Freitas et al. may be performed by controller 232.

The sentences describing the patient population may further be loaded into an embedding space, and interpreted by a machine learning system, such as an LLM deployed by controller 232. This process facilitates the identification of medical concepts that are related within the general population. For example, an LLM or regression model may calculate odds ratios and/or p-values indicating how related various reported medical concepts to each other are across the entire population.

In this embodiment, after steps 302-306 have been performed, the health server 220 has completed initialization. Specifically, now that sentences have been created describing the population of patients, the health server 220 is amenable to generating simulacrums 262 for individual subjects. For example, a subject may visit a healthcare provider and exhibit a set of non-standard phenotypes for their age or demographics. The healthcare provider may then generate a request via I/F 216 of provider client 210, requesting further insights into a cohort of patients that resemble the subject. The request is transmitted to I/F 226 via network 230. Step 308-316 describe the process of creating a simulacrum 262 in response to the request, and transmitting data describing the simulacrum 262 back to the healthcare provider (e.g., via provider client 210).

Step 308 comprises controller 232 identifying a request to generate a simulacrum for a subject. The request, received at I/F 226, includes identifying information for the subject. The request may be received as a Health Level Seven (HL7) request, an Internet-sourced request from a browser, etc. In one embodiment, controller 232 reviews the request to identify the subject within EHR data 252 and/or sequence data 240. These resources may be used by controller 232 in step 310 below. In a further embodiment, the subject is not explicitly identified, but rather indicated based on a set of medical concepts and/or phenotypes included within the request.

Step 310 comprises identifying phenotypes defining the simulacrum 262 for the subject. In some embodiments, the request explicitly indicates phenotypes that define the simulacrum 262 (e.g., by reporting specific vocabulary codes or values). In other embodiments, the request does not indicate specific phenotypes defining the simulacrum. In such instances, controller 232 may actively identify such phenotypes, such as by reviewing EHR data 252 for the subject that reports the current health of the subject. Based on the EHR data 252, controller 232 may identify medical concepts for the patient that are expected to have (or currently have) a notable impact on health or longevity of the subject. Controller 232 may then identify corresponding phenotypes associated with those medical concepts, and including those phenotypes in a definition for the simulacrum 262. In one embodiment, controller 232 references graph data structure 254, which indicates phenotypes for each of the medical concepts, in order to identify phenotypes defining the simulacrum 262.

One technique for identifying phenotypes defining the simulacrum is via a hypothesis-driven technique. In this process, the initial selection criteria for medical data and later refined/augmented criteria are selected in a supervised manner to help build cohorts. For example, a user may manually select phenotypes defining the simulacrum, or certain phenotypes may be predefined for selection based upon specific flags within EHR data for the patient. This provides a technical benefit in that it ensures that cohorts are selected in a predictable, transparent, and reproducible manner.

A further technique for identifying phenotypes defining the simulacrum is via a data-driven technique. In the data-driven technique, groups of sentences and/or patients are labeled after they are formed. For example, a group may be labeled a diabetic group based on a post hoc inspection, performed after members that are close to one and another in the latent space/embedding space are identified. In this case, when a patient or sentence has no diabetes diagnosis but sits at the center of the cluster, and assuming the clustering is robust, then it suggests something more fundamental in the biology for the patient and those in the cluster.

This in turn helps in the identification of patients or the prediction of outcomes. The phenotypes that the data represents need not be represented in a binary fashion (e.g., chosen or rejected for inclusion), but rather may be represented as weighted, fractional distributions of a collection of phenotypes, such as [0.8, 0.5, 0.1 . . . ] , the values therein mapping to a first phenotype, second phenotype, and third phenotype, respectively. In such instances, a new query or request for a simulacrum can be tested against the phenotype distributions (if using statistical methods) or tested against a corresponding embed vector (if using Machine Learning (ML) methods, where dimensions are latent features that are not mappable to clinical phenotypes one-to-one).

As used herein, phenotypes that define a simulacrum 262 are a set of phenotypes that are required, alone or in combination, for each patient to be included within a cohort that is designed to mimic the current and/or historic health of the subject. Thus, the identified phenotypes define the simulacrum 262 because they indicate the criteria for cohort selection that will be used to create the simulacrum 262. Each simulacrum 262 is therefore specific to its subject, as well as being specific to the point in time at which the request was received.

In a further embodiment, controller 232 identifies an EHR for the subject, operates an LLM (e.g., LLM 260) to identify medical concepts that currently apply to the patient, and filters the medical concepts based on their expected level of significance (e.g., as indicated by data in memory 224 associating each medical concept with a category or numeric value for significance). Filtering may comprise, for example, selecting a top N (e.g., five, ten, twenty, or fifty) medical concepts by expected level of significance (e.g., impact on health), or selecting all medical concepts applied to the patient that are within certain categories or have a numeric value for significance above a threshold for the patient. Controller 232 then includes corresponding phenotypes within the definition of the simulacrum 262.

After phenotypes that define the simulacrum 262 have been defined, controller 232 selects patients for inclusion within a cohort that mimics the current health of the subject. In one embodiment, this comprises selecting N patients from the population having combinations of sentences that most closely resemble a progression of sentences for the subject in time, and that exhibit the phenotypes defining the simulacrum. In such instances, N may comprise twenty, fifty, one hundred, one thousand, etc. In a further embodiment, controller 232 selects all patients within the population that exhibit the phenotypes defining the simulacrum. In one embodiment, controller 232 identifies patients within the population that include each of the identified phenotypes, or that include at least one identified phenotype for each of the medical concepts chosen for the simulacrum in step 310.

Step 312 comprises controller 232 retrieving sentences for a cohort of patients within the population having the identified phenotypes. Sentences from the cohort will then be used to refine the simulacrum, helping to identify potential currently undiagnosed and/or future medical concepts that may apply to the subject. Retrieving the sentences may be performed by accessing memory 224.

Step 314 comprises controller 232 identifying shared medical concepts between the patients based on the retrieved sentences. In one embodiment, controller 232 identifies shared medical concepts by identifying medical concepts that apply to patients in the cohort more frequently than in the population at-large. As a part of this process, medical concepts that were used to help define the simulacrum 262 may be excluded.

In a further embodiment, controller 232 identifies shared medical concepts by identifying medical concepts that are associated with one or more genetic variants found among the subject as well as patients within the cohort. Thus, in many embodiments, the shared medical concepts found for the simulacrum 262 represent potential outcomes for the subject, based on a bespoke cohort of patients that resemble the subject.

Step 316 comprises updating the simulacrum 262 to include the shared medical concepts. In one embodiment, controller 232 performs this action by updating simulacrum 262 in memory to report the shared medical concepts.

Step 318 comprises transmitting the simulacrum 262 toward a source of the request in response to the request. For example, in this embodiment, controller 232 may generate a command directing I/F 226 to transmit the simulacrum 262 (or a report describing the simulacrum) to provider client 210. Based on this input, a healthcare provider may initiate, adjust, or halt treatment for the subject in light of inputs described within the simulacrum.

Method 300 provides a notable benefit over prior techniques, because it provides healthcare providers with realistic, data-driven, and bespoke analyses and predictions relating to the health of specific patients at specific points in time. This facilitates the selection and implementation of preventive medical procedures which may extend the length or improve the quality of the subject's life.

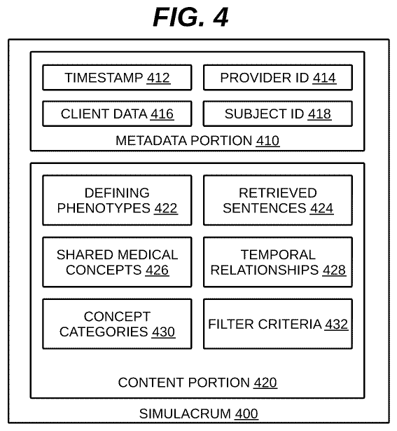

FIGS. 4-7 provide further details of illustrative components and architecture described in method 300 above. In particular, these FIGS. discuss data structures and relationships that relate to method 300.

FIG. 4 is a block diagram depicting a simulacrum 400 for a subject in an illustrative embodiment. Simulacrum 400 includes a metadata portion 410, which stores data identifying the source of a request to generate the simulacrum 400. Simulacrum 400 further includes information identifying the subject. Specifically, in this embodiment metadata portion 410 includes a timestamp 412 at which a corresponding request was received, a provider ID 414 uniquely identifying the healthcare provider (or account) that generated the request, and client data 416 indicating details about the provider client 210. Client data 416 may indicate an internet browser or EHR system used by the provider client 210, which may help controller 232 to facilitate formatting of simulacrum 400 for presentation at the provider client 210. For example, a simulacrum may be reported as a web page to an internet browser, or a custom message (e.g., a Javascript Object Notation (JSON) message) to API-driven provider clients. Metadata portion 410 further includes subject ID 418, which uniquely distinguishes the subject from other patients, such as patients within the population.

Simulacrum 400 further comprises a content portion 420. Content portion 420 includes defining phenotypes 422, which in this embodiment are stored as a list of phenotypes used for selection of patients for a cohort used to build the simulacrum 400. The list of phenotypes may be reported as medical vocabulary codes, vital measurements, etc. Content portion 420 further includes retrieved sentences 424, which may be removed after shared medical concepts for the simulacrum 400 have been determined. In this embodiment, content portion 420 additionally includes temporal relationships 428 between shared medical concepts 426, concept categories 430 used to classify a significance of the medical concepts, and filter criteria 432. Filter criteria 432 may be used to refine the cohort with additional requirements in order to ensure that the cohort only includes patients that best resemble the subject. This provides a technical benefit by enhancing the specificity of insights related to the subject.

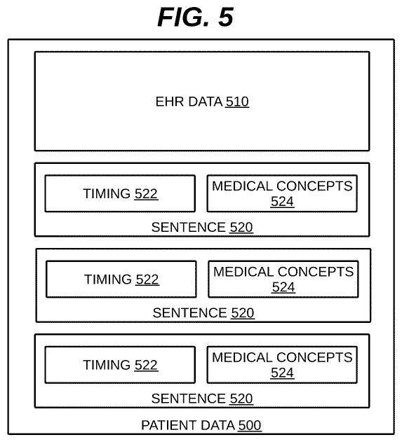

FIG. 5 is a block diagram depicting patient data 500 in an illustrative embodiment. In this embodiment, patient data 500 includes EHR data 510, such as raw EHRs and unprocessed clinical notes for the patient. Patient data 500 further includes multiple sentences 520 derived from the EHR data 510.

Each sentence 520 includes timing information 522 indicating when data for the sentence was supplied to the EHR data 510 for the patient. This may be supplied in the form of a date range, an amount of time from the birth date of the patient or a key date, etc.

Each sentence 520 further includes medical concepts 524 (e.g., as determined by LLM 260 after review of EHR data 510). Each medical concept 524 may comprise a medical vocabulary code for a medical condition, or an overarching conceptual code for a health state, such as those discussed in the OMOP Concepts vocabulary.

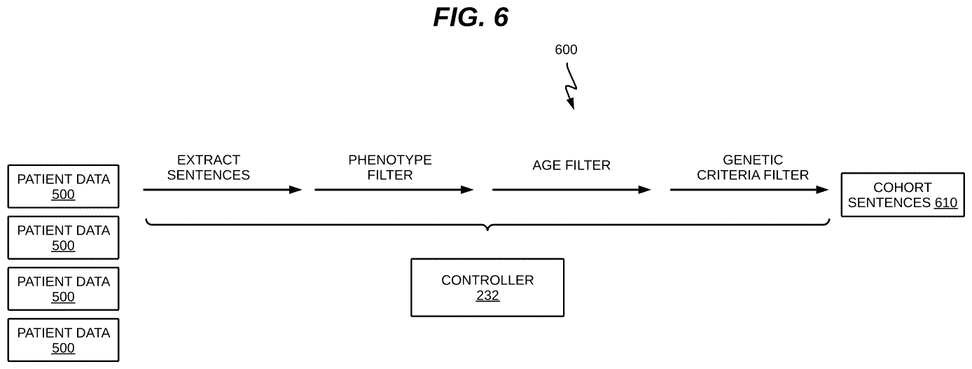

FIG. 6 is a block diagram 600 depicting processing of patient data 500 for multiple patients, in order to generate sentences for a cohort in an illustrative embodiment. As shown in FIG. 6, patient data 500 for multiple patients may be extracted into sentences for rapid processing as part of an initialization process. After initialization, the sentences for a group of patients may be filtered based on phenotypes defining the simulacrum, such that patients are excluded if their sentences do not report the phenotypes. The patients may then be further filtered based on similarity of an age at which the phenotypes were detected for those patients to an age at which the subject expressed the phenotypes, and/or based on genetic criteria. Genetic criteria may include, for example, a requirement that a patient have the same or a similar genetic variant to the subject. After filters have been applied, the cohort sentences 610 which remain are used by controller 232 to build a simulacrum 400 for the subject.

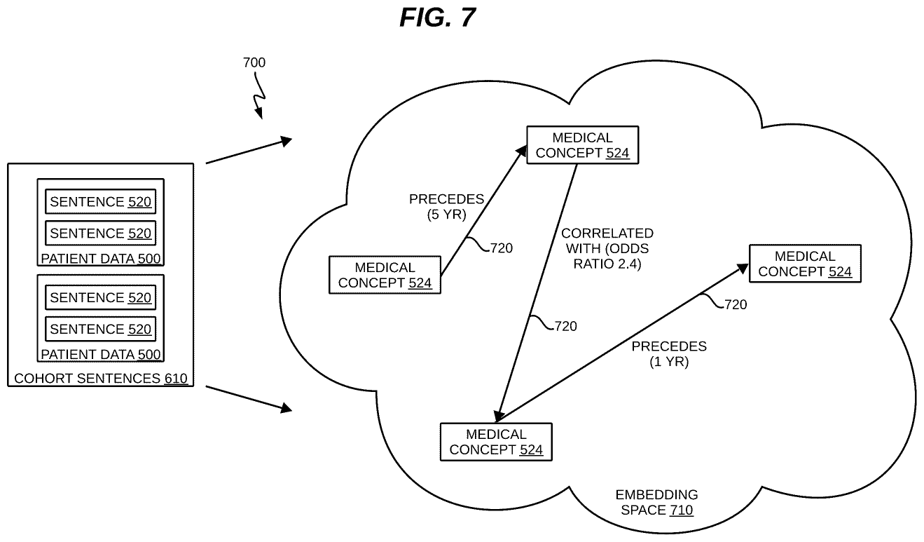

FIG. 7 is a block diagram 700 depicting relations between medical concepts 524 reported within sentences 520 for a cohort in an illustrative embodiment. In this embodiment, distances between sentences are measured within an embedding space 710 in order to identify relationships 720 between them. The embedding space 710 may comprise, for example, one or many thousands of dimensions. Embedding of sentences 520 within the embedding space may be performed via an algorithm such as Word2vec, Phe2vec, etc., such as described in De Freitas et al, or via other well understood embedding techniques for data. Distances within the embedding space 710 may then be utilized to anticipate temporal and/or correlative relationships between medical concepts 524 and or sentences 520.

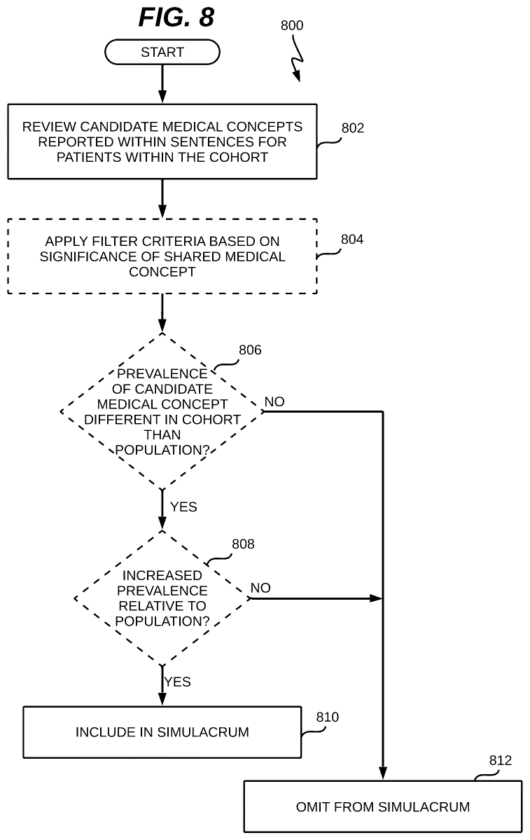

FIGS. 8-11 describe various enhancements, alternatives, and/or refinements to method 300 in illustrative embodiments.

FIG. 8 is a flowchart depicting a method 800 for selectively deciding whether to include medical concepts in a simulacrum, based on a prevalence of the medical concepts within a cohort of patients in an illustrative embodiment.

Step 802 comprises controller 232 reviewing candidate medical concepts reported within sentences for patients within the cohort. As used herein, candidate medical concepts are medical concepts that are exhibited by at least one patient in the cohort. However, candidate medical concepts may have a negligible impact on patient health, or may occur with similar frequency to the general population. Thus, the steps described herein facilitate the detection of insights that are both relevant to the subject and insightful in relation to the health of the subject.

In step 804, controller 232 applies filter criteria based on the significance of each candidate medical concept. For example, controller 232 may filter out or otherwise remove shared medical concepts that are below a threshold level of significance in relation to patient health. A threshold level of significance may be met by serious medical conditions defined by the Family Medical Leave Act (FMLA) (comprising an illness, injury, impairment, or physical or mental condition which requires overnight hospitalization or continuing treatment of an extended period of time and episodic periods of incapacity), complex medical conditions that involve multiple organ systems and are typically chronic in nature, medical conditions which are expected to notably reduce either the duration or quality of life of a patient, etc. In further embodiments, medical concepts are each associated with a significance score within memory 224, and the threshold for significance is predetermined. In still further embodiments, controller 232 selects the top N candidate medical concepts having the highest significance score (e.g., where N is five, ten, or twenty). In some embodiments, step 804 may be performed after step 808, which may help for example to ensure that simulacrums created for different patients include a roughly uniform number of shared medical concepts.

Step 806 comprises controller 232, for each candidate medical concept, comparing a prevalence within the cohort to a prevalence within the population. This may be determined by calculating an odds ratio or p-value that the occurrence rate for the candidate medical concept (e.g., as shown by occurrence rates for corresponding phenotypes reported within EHR data) within the cohort is different than in the population. If the prevalence is different, and the confidence in this determination is high as indicated for example by a p-value (e.g., below 0.1, below 0.05, or below 0.01) or an odds ratio (e.g., above 1, above 2, or above 5) then processing continues to step 808. Otherwise processing continues to step 812 and the candidate medical concept is omitted from the simulacrum 400 for the patient.

Step 808 comprises controller 232 determining whether the prevalence of the candidate medical concept within the cohort is increased relative to the population. In one embodiment, if the prevalence is higher, then processing continues to step 810 and the candidate medical concept is included within the simulacrum 400. Otherwise, processing continues to step 812 and the candidate medical concept is omitted. In one embodiment, higher prevalence is indicated by a higher per-capita rate within the cohort than the population, potentially modified to account for age and/or other differences between the cohort and the population.

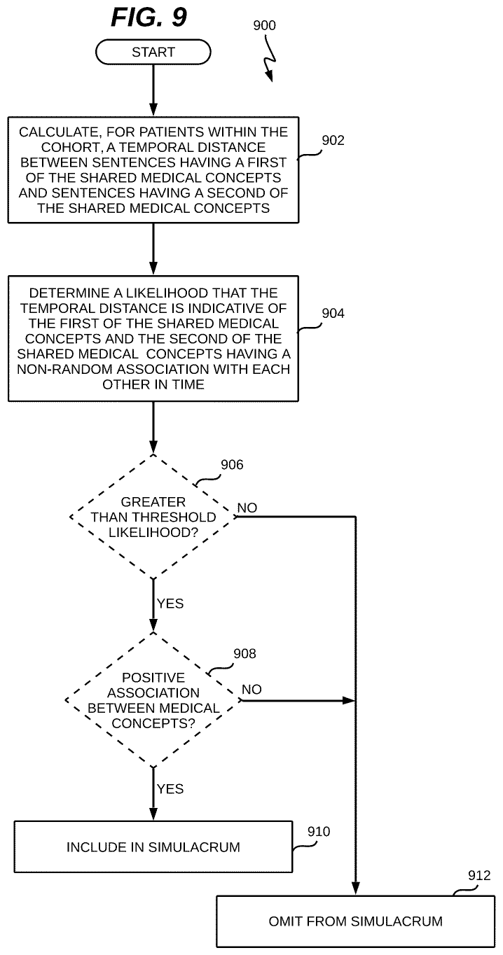

FIG. 9 is a flowchart depicting a method 900 for identifying temporal relationships between medical concepts in a simulacrum, in an illustrative embodiment. As used herein, a temporal relationship is a non-random relationship in time between two sentences, and/or two or more medical concepts reported within sentences. These relationships include causal relationships and/or time-based correlative relationships. For example, a temporal relationship may comprise a mean or median time between diagnosis of a first medical concept and a second medical concept within the cohort assembled for a subject.

In step 902, controller 232 calculates a temporal distance between sentences having a first of the shared medical concepts and sentences having a second of the shared medical concepts. This may comprise determining a typical (e.g., median, or mean) distance in time between the sentences and/or the medical concepts, in real-time or within the embedding space. As used herein, temporal distances are not absolute values, but can indicate negative distances (backward in time) as well as positive distances (forward in time), with the caveat that negative distance relationships can be rewritten as forward distance relationships. That is, a relationship that A tends to precede B may be rewritten as a relationship that B tends to occur after A.

In step 904, controller 232 determines a likelihood that the temporal distance is indicative of the first of the shared medical concepts and the second of the shared medical concepts having a non-random association in time. This may be achieved by calculating a confidence level (e.g., an odds ratio or p-value) that the first and second shared medical concepts are related in time, and then ensuring that the confidence level is higher than a threshold (e.g., as shown by a p-value that is less than 0.1, 0.05, or 0.01, or an odds ratio that is greater than 1, 2, or 5).

If the likelihood is greater than the threshold value in step 906, then processing continues to step 908. Otherwise, processing continues to step 912 and the temporal relationship is omitted from the simulacrum 400.

In step 908, controller 232 determines whether there is a positive association between the first shared medical concept and the second shared medical concept. A positive association exists if the presence of one of the medical concepts increases the likelihood of another of the medical concepts occurring. In this embodiment, in the event that a positive association exists, the temporal relationship is included in the simulacrum 400 in step 910. Otherwise, processing continues to step 912 and the temporal relationship is omitted. This step may be performed, for example, to focus content for the simulacrum 400 upon identifying and addressing potential future medical conditions of the patient.

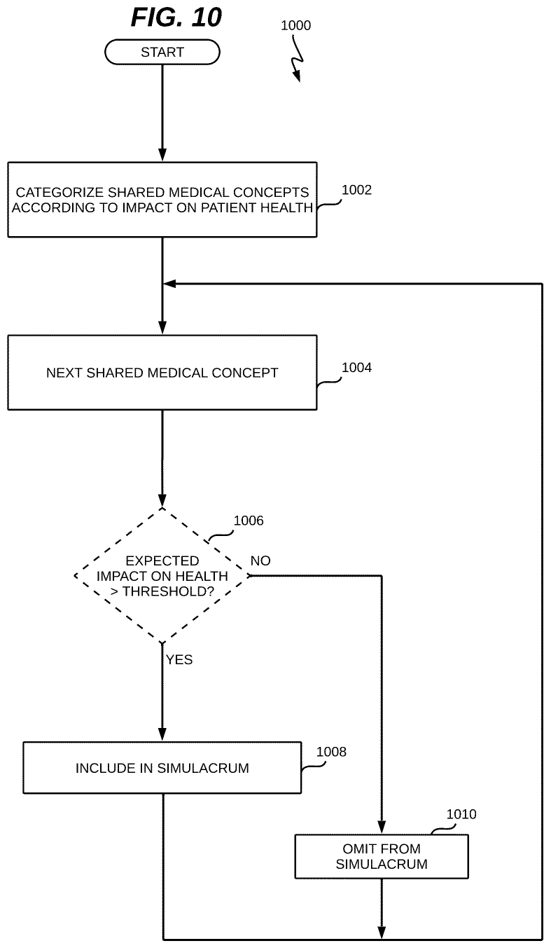

FIG. 10 is a flowchart depicting a method 1000 for categorization of medical concepts for selective inclusion within a simulacrum, in an illustrative embodiment. Method 1000 may be utilized, for example, to filter medical concepts based on their expected impact on the health of the subject.

In step 1002, controller 232 categorizes shared medical concepts according to impact on patient health. For example, controller 232 may consult predetermined categorizations for each medical concept. Examples of categories include serious medical conditions defined by the Family Medical Leave Act (FMLA) (comprising an illness, injury, impairment, or physical or mental condition which requires overnight hospitalization or continuing treatment of an extended period of time and episodic periods of incapacity), complex medical conditions that involve multiple organ systems and are often chronic in nature, medical conditions which are expected to notably reduce either the duration or quality of life of a patient, etc.

In step 1004, controller selects a next shared medical concept for review. If in step 1006 the expected impact on health is greater than a threshold amount (e.g., as indicated by a specific categorization or a numerical value for impact on health), then the shared medical concept is included in the simulacrum 400 in step 1008. Otherwise, the shared medical concept is omitted from the simulacrum 400 in step 1010. A next shared medical concept is selected and reviewed in an iterative process, until all shared medical concepts within the cohort for the simulacrum have been reviewed.

Filtering via method 1000 provides a technical benefit by eliminating medical conditions that are not considered relevant or critical by the healthcare provider that generated the request. This helps to ensure that the healthcare provider can efficiently review the simulacrum in a limited amount of time while still identifying those medical conditions and concepts most likely to have an impact on the health of the subject.

In further embodiments, medical concepts include genetic testing results, and graph data structure 254 includes medical concepts for genetic testing results, such as variant classifications, carrier status for pathogenic variants, and other reporting information, on a patient-by-patient basis for patients reported by the EHR data 252. Hence, in some embodiments graph data structure 254 exhibits an even more unique architecture, in the form of a combination of nodes that consider both EHR-linked medical concepts and genetic testing-linked medical concepts. This enables a user to build a cohort of patients not just by reference to EHR data 252, but also by reference to sequence data 240. In short, this arrangement provides a notable technical benefit by permitting selection of a cohort of patients based on clinicogenomic criteria.

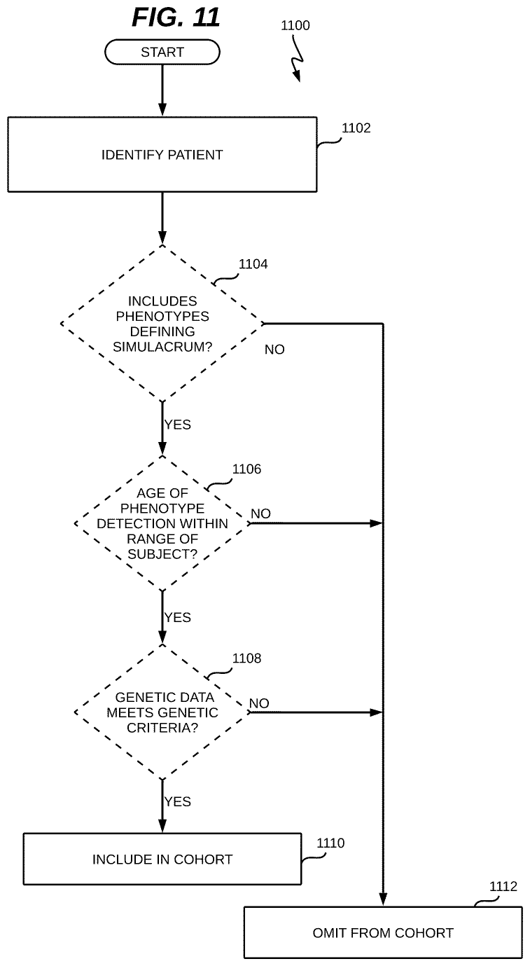

FIG. 11 is a flowchart depicting a method 1100 for selectively deciding whether to include patients within a cohort, based on age and/or genetic criteria in an illustrative embodiment.

Step 1102 comprises controller 232 identifying a patient for review, such as any patient within the population. Step 1104 comprises controller 232 determining whether EHR data 252 for the patient includes phenotypes defining the simulacrum 400 for the subject. In one embodiment, each phenotype defining the simulacrum 400 is required in order for a patient to be included within the cohort. Therefore, at least one clinical note or other data point (e.g., a medical vocabulary code, measurement, treatment, procedure, etc.) within EHR data 252 or sentences 520 must exist for the patient for each phenotype in the simulacrum 400, in order for the patient to be included in the cohort.

In a further embodiment each phenotype defining the simulacrum 400 is one of multiple phenotypes associated with a medical concept defining the simulacrum 400. In such circumstances, in one embodiment controller 232 requires that at least one phenotype for each medical concept defining the simulacrum 400 be present in EHR data 252 for the patient, in order for the patient to be included in the cohort.

In the event that data for the patient includes phenotypes defining the simulacrum in step 1104, processing continues to step 1106. Otherwise, the patient is omitted from the cohort in step 1112.

In step 1106, the controller 232 determines whether an age of the patient at first detection of a defining phenotype is within a desired range for the subject. This may comprise phenotype detection ages within a threshold number of years of a phenotype detection age for the patient (e.g., five years, ten years, or twenty years), an explicit exclusion on patients that do not fall within the same age group as the patient at the time of phenotype detection, (e.g., age groups of youth ages zero to seventeen, adult ages eighteen to fifty nine, and senior ages sixty and up), etc. In further embodiments, the age-based screening is performed for each phenotype and/or medical concept defining the patient, or for a subset of such phenotypes and/or medical concepts (e.g., as defined by a user).

In an event that the phenotype detection ages of the patient are within the range desired for the subject, processing continues onward to step 1108. Otherwise, processing continues to step 1112 and the patient is omitted from the cohort.

In step 1108, controller 232 determines whether genetic data for the patient meets genetic criteria defined for the simulacrum. The genetic criteria defined for the simulacrum may comprise one or more variants, or categories of variants, exhibited by the subject. In one embodiment, genetic data is reviewed by controller 232 in the form of VCF or other data formats, in order to determine whether the patient qualifies. Example genetic criteria include a need for a Loss of Function (LoF) variant or coding variant within a specific gene, at a specific locus or set of loci, or within a set of genes and/or loci associated with a medical concept exhibited by the patient (e.g., a variant associated with Polycystic Kidney Disease (PKD), or another disease with a genetic association).

If the patient meets the genetic criteria, the patient is included in the cohort in step 1110. Otherwise, the patient is omitted from the cohort by controller 232 in step 1112.

While step 1108 is integrated with steps 1104 and 1106 in method 1100, in further embodiments one or more of these steps are not required for use together, and/or may be omitted entirely.

In further embodiments, patients are selectively included by controller 232 within the cohort. This may be performed by selecting a number N of patients that most closely resemble the patient in terms of phenotype, medical concept, age of phenotype detection, and/or sentences. For example, the closest twenty, one hundred, or one thousand patients may be selected for the cohort, rather than using the strict filtering process described above. This may provide a technical benefit by ensuring that patients with rare conditions still have a sufficient cohort size from which to draw insights.

In further embodiments, the building of cohorts may be validated with, or supplemented by, the process of vectorizing patient information, such as via the processes described in “Phe2vec: Automated disease phenotyping based on unsupervised embeddings from electronic health records,” De Freitas, Jessica K. et al., Patterns, Volume 2, Issue 9, 100337, herein incorporated by reference. Comparison of patients within a vectorized space may facilitate the identification of similar patients in a manner that helps to ensure the accuracy and breadth of the graph data structure techniques described herein.

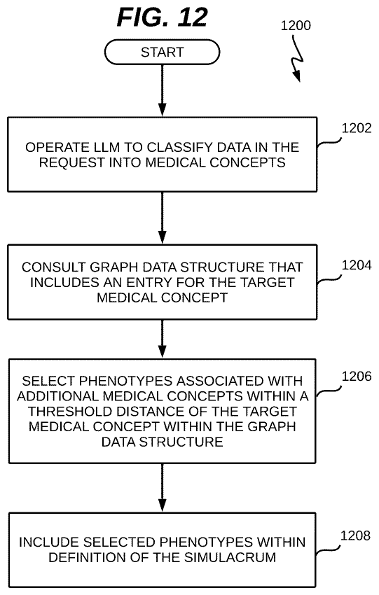

FIGS. 12-17 describe illustrative embodiments wherein a controller 232 consults a graph data structure to facilitate the steps of method 300 in illustrative embodiments. For example, FIGS. 12-17 may be utilized to facilitate the translation of EHR data such as metrics or text into medical concepts in step 306 of method 300, to identify phenotypes associated with specific medical concepts in step 310 of method 300, etc.

FIG. 12 is a flowchart depicting a method 1200 for processing natural language input in an illustrative embodiment. Method 1200 may be performed, for example, during steps 308-310 of method 300.

Assume, for that embodiment, that I/F 226 receives natural language input as a part of a query from the user. For example, the natural language input may comprise plain text or rich text, stored within a text field of the request. The natural language input includes text referring to one or more medical concepts, but need not refer to specific medical vocabulary codes, measurements, laboratory results, diseases or conditions associated with those medical concepts. The natural language may be included, for example, within a portion of the request used to help define, in plain language, key aspects of the simulacrum being constructed.

Step 1202 comprises controller 232 operating LLM 260 to classify data in the request into one or more medical concepts. This may comprise, for example, controller 232 instructing LLM 260 to identify natural language referring to medical concepts, or may comprise correlating vocabulary codes recited within the request with specific medical concepts.

Controller 232 may operate LLM 260 to identify multiple medical concepts within the same natural language. In a further embodiment, controller 232 instructs LLM 260 to identify not just medical concepts, but also values associated with those concepts. For example, a medical concept relating to high blood pressure may be assigned a desired range of values of greater than 120 mmHg within the natural language input. The LLM 260 therefore operates to identify the corresponding value within the natural language input, and to associate it with the medical concept.

Step 1204 comprises consulting a graph data structure 254 that includes entries for the medical concepts from the request. Controller 232 may instruct LLM 260 to compare medical concepts from the request to nodes in the graph data structure 254. LLM 260 seeks out medical concepts from the request that have a high confidence relationship to a specific entry or node within the graph data structure 254. In one embodiment, this comprises LLM 260 comparing words and phrases within natural language to words and phrases recited in medical concepts. In the event that the comparison results in more than a threshold level of confidence (e.g., self-reporting by the LLM 260 of a “high” level of confidence that a phrase is the same as a medical concept reported in a node), the medical concept from the request is confirmed for use with the simulacrum. In a further embodiment, the LLM 260 vectorizes the natural language and/or phrase being considered, and controller 232 compares this vectorized content to vectorized versions of medical concepts maintained in memory 224.

Step 1206 comprises controller 232 operating the LLM 260 to select phenotypes associated with additional medical concepts that are within a threshold distance of each medical concept within the graph data structure 254 that was selected for use with the simulacrum. This may be performed, for example, by counting the smallest number of edges between a node for the medical concept selected for use with the simulacrum and a node describing a phenotype. In one embodiment, each node describes both a medical concept and phenotypes. Controller 232 may select phenotypes recited for medical concepts that are within a threshold distance of the medical concept selected for use with the simulacrum.

Step 1208 comprises including the selected phenotypes within a definition of the simulacrum. Controller 232 may include selected phenotypes by adding the phenotypes to within a content portion of the simulacrum, using numeric metrics, vocabulary codes, plain text, or other flags which facilitate detection of the selected phenotypes from sentences and/or EHR data 252.

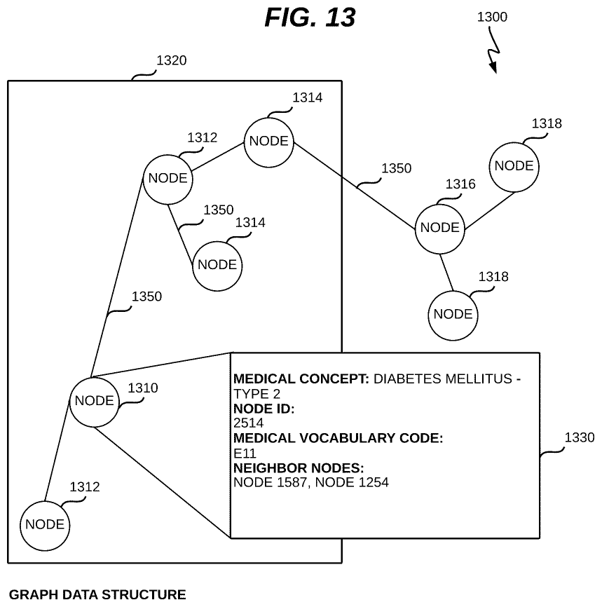

FIG. 13 depicts a graph data structure 1300 in an illustrative embodiment. Graph data structure 1300 is a simplified version of a graph data structure 254, provided to enhance conceptual understanding of nodes, the contents of nodes, and edges between nodes. In this embodiment, graph data structure 1300 includes a node 1310 which corresponds with a medical concept identified within a query. Controller 232 selects all nodes within a threshold distance of two steps of the node 1310. This means that nodes 1312 that are one step from node 1310, and nodes 1314 that are two steps from node 1310, are included within selection criteria 1320. Node 1316 and nodes 1318 are not selected. Distances are determined by counting the number of edges 1350 between nodes. In this embodiment, each node includes its own content 1330, which stores information identifying neighbor nodes, identifying the current node, and reciting a medical vocabulary code, medical concept, laboratory test, and/or measurement for the node.

Graph data structure 1300 provides a unique architecture for storing medical concept data, by tying specific nodes and concepts to specific types of EHR-linked content. This enables a concept to be directly mapped to specific portions of EHR data, even portions of EHR data that are maintained within free-text fields. This facilitates the operation of an LLM to identify desired portions of content that correspond to specific medical concepts within EHR data 252.

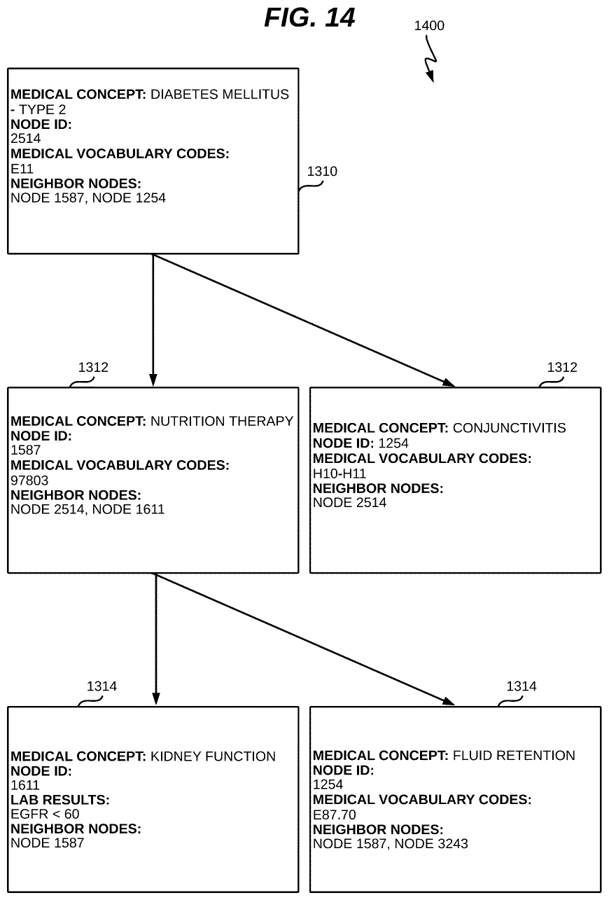

FIG. 14 depicts a selection 1400 of nodes within a graph data structure in an illustrative embodiment. Specifically, FIG. 14 depicts the same selection of nodes shown in FIG. 13 for graph data structure 1300. As shown in FIG. 14, nodes 1312 are within one step of node 1310, and nodes 1314 are within two steps of node 1310. Each node includes its own content reciting information relevant to a medical concept of interest, including for example medical vocabulary codes and phenotype data.

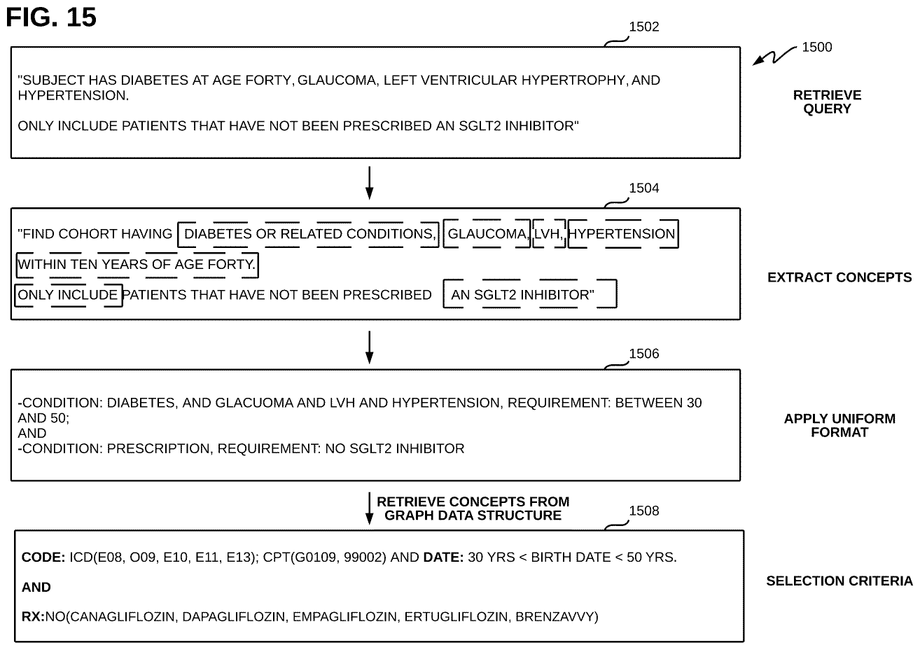

FIG. 15 is a diagram 1500 that depicts processing of natural language content from a request in an illustrative embodiment. As shown in FIG. 15, in one embodiment natural language 1502 is retrieved from a request by an LLM 260. The LLM extracts concepts from the natural language by phrases 1504 that correspond with medical conditions, measurements, or laboratory procedures. The LLM then processes the extracted concepts into a uniform format 1506. In this embodiment, the uniform format indicates the name of each concept, followed by values required for each concept. Next, the LLM 260 retrieves the concepts from the graph data structure to generate a set of selection criteria 1508. Selection criteria 1508 may comprise, for example a set of phenotypes used to define the simulacrum for the subject.

In a further embodiment, the extraction of medical concepts is performed via a pretrained Non-Linear Programming (NLP) model, such as Amazon Web Services (AWS) comprehend medical, or by a an LLM 260 that accesses the NLP model as a tool). AWS comprehend is using a pretrained NLP model (a deep learning based one but not a generative model).

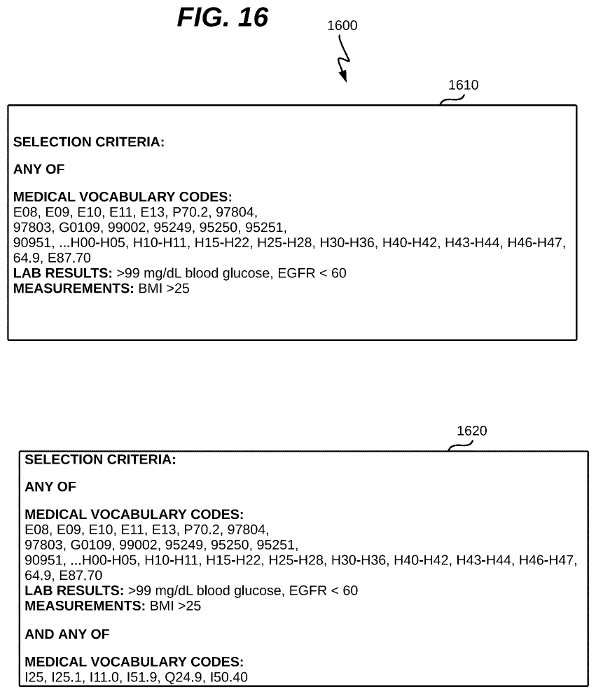

FIG. 16 is a block diagram 1600 depicting selection criteria for a cohort in an illustrative embodiment. Specifically, FIG. 16 depicts selection criteria 1610 as well as selection criteria 1620. Selection criteria 1610 indicates that any patient having a specific medical vocabulary code, laboratory result (e.g., prior to receiving medication related to the medical concept), or measurement (e.g., prior to receiving medication related to the medical concept) qualifies for the cohort.

Selection criteria 1620 selects only patients that have both one of a wide range of medical codes, as well as one of a narrow range of medical codes. This facilitates precision cohort selection.

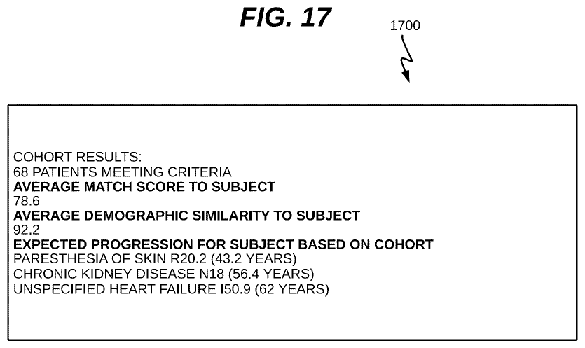

FIG. 17 is a block diagram 1700 that depicts a cohort summary in an illustrative embodiment. The cohort summary recites a number of patients in the cohort, an average matching score of patients in the cohort to the subject, an average demographic similarity of patients in the cohort, and a list of expected medical concepts and corresponding ages expected for the subject based on the simulacrum. The matching score for patients may be determined by calculating a distance in an embedding space between sentences exhibiting defining phenotypes for patients in the cohort, and sentences defining corresponding phenotypes for the subject. The score may then be calculated as an inverse of the distance, and/or be scaled to a bounded numeric range (e.g., between zero and one hundred). The demographic similarity for subjects may be determined by comparing a predetermined set of demographic characteristics between the subject and the patients in the cohort. These demographics may include ages of presentation for each defining phenotype, sex assigned at birth, ancestry, and other demographics.

The expected progression may be determined by listing shared medical concepts identified within the cohort that have not yet been presented by the subject (e.g., within EHR data 252 or sentences for the subject), and then applying temporal relationships to anticipate the age of presentation of phenotypes for these medical concepts.

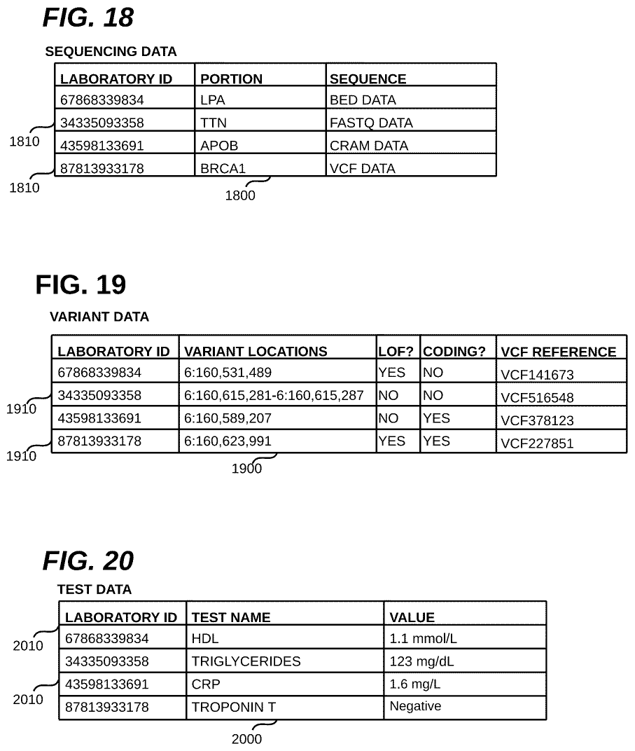

FIG. 18 is a table 1800 that summarizes sequencing data for one or more genes for individuals in an illustrative embodiment. For example, table 1800 may be one of many data structures stored in health server 220. In this embodiment, table 1800 includes an entry 1810 for each of multiple patients. Each entry 1810 includes a unique identifier (e.g., LSID) for the corresponding patient, as well as an indication of the gene that the sequence data relates to. The portion of the genome that has been sequenced may comprise whole genome data, whole exome data, array data, data for a specific gene or portion of a gene, etc. Table 1800 also indicates a format of the sequence data. Table 1800 may be generated based on, or with reference to, sequences that have been alignment-enhanced via the processes discussed above.

FIG. 19 is a table 1900 that summarizes variant data for individuals in an illustrative embodiment. In this embodiment, each entry 1910 in table 1900 reports a location (e.g., chromosomal coordinate) for each genetic variant, together with flags indicating whether the variant is a Loss of Function (LoF) variant or a coding variant. Table 1900 further includes a VCF reference, which refers to the location and/or identifier of a VCF file that indicates the presence of the variant. The VCF file may be generated using data from the alignment enhancement processes discussed above. For example, alignment-enhanced data in a BAM, SAM, or CRAM file may include data used to generate the VCF file. Table 1900 may be utilized by controller 232 of health server 220, in order to rapidly select and report diagnostic and treatment thresholds for a patient. Table 1900 may be generated based on, or with reference to, sequences that have been alignment-enhanced via the processes discussed above.

FIG. 20 is a table 2000 that summarizes biomarker test data for individuals in an illustrative embodiment. Specifically, table 2000 summarizes test data pertaining to predetermined diseases for each of multiple patients in an illustrative embodiment. Each entry 2010 in table 2000 indicates an anonymized laboratory ID for a patient, a corresponding test name, and a corresponding value. Table 2000 may be created, for example, based on EHR data retrieved for patients. Laboratory IDs may be associated with EHR identifiers at health server 220 or provider client 210, in order to enable access to both health data and genomics data for a patient. Table 2000 may be used to enhance or provide context for genetic insights determined based sequences that have been alignment-enhanced via the processes discussed above.

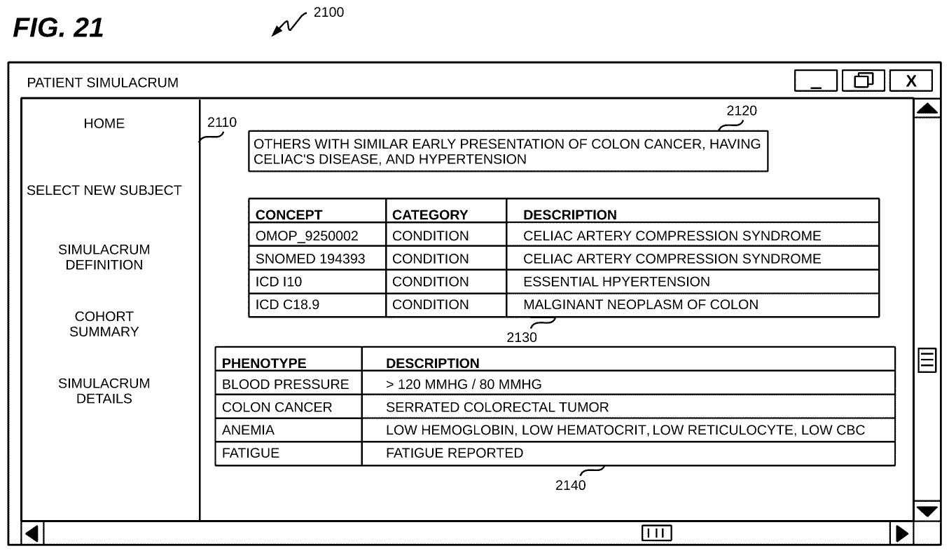

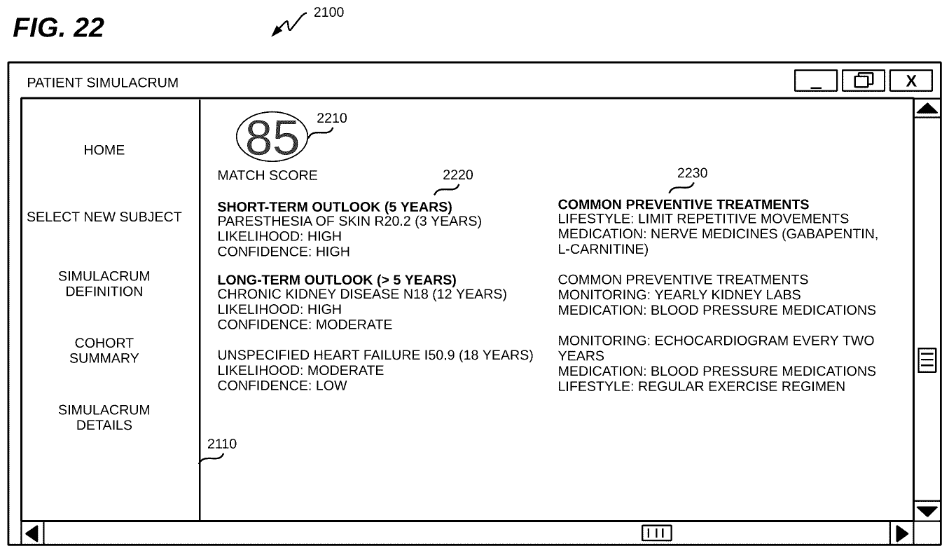

FIGS. 21-22 depict Graphical User Interfaces (GUIs) that facilitate the creation of simulacrums for subjects in illustrative embodiments. These GUIs may be presented, for example, via a browser window or other portion of a screen of one or more provider clients 210.

FIG. 21 depicts a GUI 2100 for selecting subjects, defining a simulacrum for a selected subject, reviewing a summary of a cohort chosen for a subject, and reviewing details for a created simulacrum for a subject in an illustrative embodiment. For example, GUI 2100 may be implemented via provider client 210 in order to facilitate user operations pertaining to simulacrums 262.

In this embodiment, GUI 2100 includes a menu portion 2110. Menu portion 2110 provides access to multiple screens of GUI 2100. These screens include a home screen, which provides access to prior-generated simulacrums for subjects, a selection screen which permits the user to select a subject (e.g., via a unique ID for the patient used by the healthcare provider, such as a Medical Record Number (MRN) or similar), a simulacrum definition screen which permits the user to adjust a simulacrum definition that has been either automatically or manually generated, a cohort summary describing patients from the population that meet the simulacrum definition, and simulacrum details describing common conditions and expected outcomes for the subject that the simulacrum was generated for.

As depicted, GUI 2100 displays a simulacrum definition screen, which includes a natural language field 2120, as well as a medical concept list 2130 and a set of defining phenotypes 2140. The medical concept list 2130 may be determined by reference to natural language field 2120 (e.g., as analyzed by LLM 260) and/or EHR data 252 for the subject. Similarly, the defining phenotypes 2140 may be determined by reference to EHR data 252, natural language field 2120, and/or medical concept list 2130.

FIG. 22 depicts the GUI 2100 of FIG. 21, this time displaying a simulacrum details screen. In FIG. 22, the simulacrum details screen includes a matching score 2210, which indicates a degree to which patients in the cohort match the demographics (e.g., ancestry and/or age) of the subject. In further embodiments, the matching score further comprises a degree to which shared medical concepts within the cohort are consistently found within the cohort.

The simulacrum details screen further includes an outlook portion 2220 and an insights portion 2230. The outlook portion 2220 recites shared medical concepts found within the cohort that have not been already listed in EHR data 252 for the subject. Thus, the outlook portion 2220 may recite undiagnosed, or yet-to-be manifested medical concepts. In this embodiment, the outlook portion is segmented into a short-term outlook and long-term outlook. Each outlook indicates shared medical concepts expected for the subject, a vocabulary code for each shared medical concept, a likelihood that the subject will experience the shared medical concept (based on prevalence within the cohort), and timing information indicating when the shared medical concept is expected to be experienced by the subject. In this embodiment, each shared medical concept is also accompanied by a confidence value. This may indicate, based on the degree of matching between the cohort and the subject, an amount of confidence in the outlook.

The insights portion 2230 is tailored to the shared medical concepts recited in the simulacrum, and may comprise a list of best practices to detect, prevent, and/or treat each of the shared medical concepts. For example, these best practices may be drawn from medical literature associated with each shared medical concept, and/or may be stored in memory 224. Health server 220 may therefore report this information back to provider client 210 when responding to the request for the simulacrum. Insights portion 2230 provides a technical benefit by helping to ensure that healthcare providers have an actionable real-world understanding of next steps that could be taken to enhance the health of subjects.

Any of the various computing and/or control elements shown in the figures or described herein may be implemented as hardware, as a processor implementing software or firmware, or some combination of these. For example, an element may be implemented as dedicated hardware. Dedicated hardware elements may be referred to as “processors,” “controllers,” or some similar terminology. When provided by a processor, the functions may be provided by a single dedicated processor, by a single shared processor, or by a plurality of individual processors, some of which may be shared. Moreover, explicit use of the term “processor” or “controller” should not be construed to refer exclusively to hardware capable of executing software, and may implicitly include, without limitation, digital signal processor (DSP) hardware, a network processor, application specific integrated circuit (ASIC) or other circuitry, field programmable gate array (FPGA), read only memory (ROM) for storing software, random access memory (RAM), non-volatile storage, logic, or some other physical hardware component or module.

In one embodiment, instructions stored on a computer readable medium direct a computing system of any of the devices and/or servers discussed herein, such as health server 220, to perform the various operations disclosed herein. In some embodiments, all or portions of these operations may be implemented in a networked computing environment, such as a cloud computing system. Cloud computing often includes on-demand availability of computer system resources, such as data storage (cloud storage) and computing power, without direct active management by an entity. Cloud computing relies on the sharing of resources, and generally includes on-demand self-service, broad network access, resource pooling, rapid elasticity, and measured service.

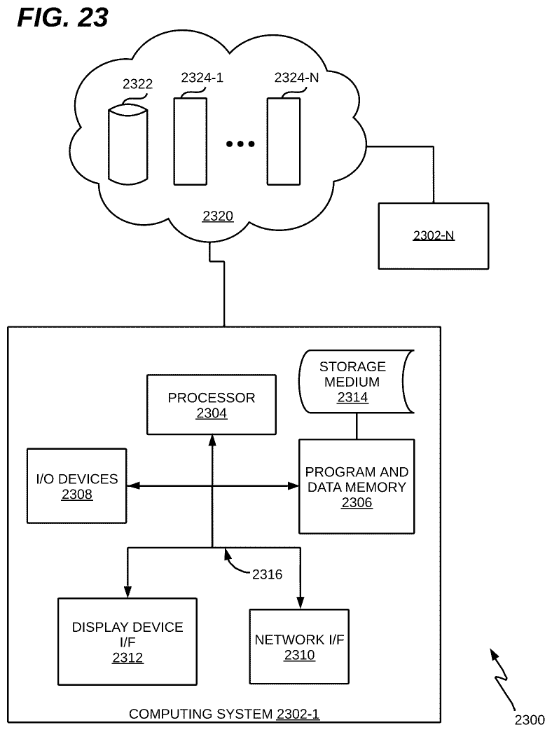

FIG. 23 depicts one illustrative cloud computing system 2300 operable to perform the above operations by executing programmed instructions tangibly embodied on one or more computer readable storage mediums. The cloud computing system 2300 generally includes the use of a network of remote servers hosted on the internet to store, manage, and process data, rather than a local server or a personal computer (e.g., in the computing systems 2302-1-2302-N). Cloud computing enables users to use infrastructure and applications via the internet, without installing and maintaining them on-premises. In this regard, the cloud computing network 2320 may include virtualized information technology (IT) infrastructure (e.g., servers 2324-1-2324-N, the data storage module 2322, operating system software, networking, and other infrastructure) that is abstracted so that the infrastructure can be pooled and/or divided irrespective of physical hardware boundaries. In some embodiments, the cloud computing network 2320 can provide users with services in the form of building blocks that can be used to create and deploy various types of applications in the cloud on a metered basis.

Various components of the cloud computing system 2300 may be operable to implement the above operations in their entirety or contribute to the operations in part. For example, a computing system 2302-1 may be used to perform analysis of gene sequencing data, and then store that analysis along with the gene sequencing data in a data storage module 2322 (e.g., a database) of a cloud computing network 2320. Various computer servers 2324-1-2324-N of the cloud computing network 2320 may be used to operate on the gene sequencing data and/or transfer the gene sequencing analysis and/or the gene sequencing data to another computing system 2302-N.

Some embodiments disclosed herein may utilize instructions (e.g., code/software) accessible via a computer-readable storage medium for use by various components in the cloud computing system 2300 to implement all or parts of the various operations disclosed hereinabove. Examples of such components include the computing systems 2302-1-2302-N.

Exemplary components of the computing systems 2302-1-2302-N may include at least one processor 2304, a computer readable storage medium 2314, program and data memory 2306, input/output (I/O) devices 2308, a display device interface 2312, and a network interface 2310. For the purposes of this description, the computer readable storage medium 2314 comprises any physical media that is capable of storing a program for use by the computing system 2302. For example, the computer-readable storage medium 2314 may be an electronic, magnetic, optical, electromagnetic, infrared, semiconductor device, or other non-transitory medium. Examples of the computer-readable storage medium 2314 include a solid-state memory, a magnetic tape, a removable computer diskette, a random access memory (RAM), a read-only memory (ROM), a rigid magnetic disk, and an optical disk. Some examples of optical disks include Compact Disk-Read Only Memory (CD-ROM), Compact Disk Read/Write (CD-R/W), Digital Versatile Disc (DVD), and Blu-Ray Disc.

The processor 2304 is coupled to the program and data memory 2306 through a system bus 2316. The program and data memory 2306 include local memory employed during actual execution of the program code, bulk storage, and/or cache memories that provide temporary storage of at least some program code and/or data in order to reduce the number of times the code and/or data are retrieved from bulk storage (e.g., a hard disk drive, a solid state drive, or the like) during execution.

Input/output or I/O devices 2308 (including but not limited to keyboards, displays, touchscreens, microphones, pointing devices, etc.) may be coupled either directly or through intervening I/O controllers. Network adapter interfaces 2310 may also be integrated with the system to enable the computing system 2302 to become coupled to other computing systems or storage devices through intervening private or public networks. The network adapter interfaces 2310 may be implemented as modems, cable modems, Small Computer System Interface (SCSI) devices, Fibre Channel devices, Ethernet cards, wireless adapters, etc. Display device interface 2312 may be integrated with the system to interface to one or more display devices, such as screens for presentation of data generated by the processor 2304.

What is claimed is:

1. A system for anticipating potential medical conditions for patients, the system comprising:
a controller configured to retrieve Electronic Health Record (EHR) data for each patient within a population, to partition the EHR data for each patient into discrete time periods, and for each time period for each patient, to assemble a sentence comprising a combination of medical concepts for the patient during the time period, resulting in conversion of EHR data for that patient into multiple sentences;
the controller is further configured to identify a request to generate a simulacrum for a subject, to identify phenotypes defining the simulacrum, to retrieve sentences for a cohort of patients within the population having the identified phenotypes, to identify shared medical concepts between the patients based on the retrieved sentences, and to update the simulacrum to include the shared medical concepts;
the controller further configured to generate a command to share the simulacrum in response to the request; and
an interface configured to transmit the simulacrum toward a source of the request.

2. The system of claim 1 wherein:
the controller is further configured to identify temporal relationships between the shared medical concepts, and update the simulacrum to include the temporal relationships.

3. The system of claim 2 wherein:
the temporal relationships comprise a mean or median time between diagnosis of a first of the shared medical concepts and a second of the shared medical concepts.

4. The system of claim 2 wherein:
the controller is further configured to identify temporal relationships between the shared medical concepts by:
calculating, for patients within the cohort, a temporal distance between sentences having a first of the shared medical concepts and sentences having a second of the shared medical concepts; and
determining a likelihood that the temporal distance is indicative of the first of the shared medical concepts and the second of the shared medical concepts having a non-random association with each other in time.

5. The system of claim 1 wherein:
the controller is further configured to identify the shared medical concepts by:
reviewing candidate medical concepts reported within sentences for patients within the cohort;
for each of the candidate medical concepts: comparing a prevalence within the cohort to a prevalence within the population; and
identifying a candidate medical concept as a shared medical concept in response to determining that a prevalence of the candidate medical concept is greater in the cohort than in the population.

6. The system of claim 1 wherein:
the controller is further configured to categorize medical concepts based on expected impact to patient health, and to filter contents of the simulacrum to exclude shared medical concepts having less than a threshold impact on patient health.

7. The system of claim 1 wherein:
the controller dynamically assembles the cohort for the patient by excluding patients having an age of presentation for at least one of the phenotypes which differs by more than a threshold amount from an age of presentation for the subject.

8. The system of claim 1 wherein:
the controller is further configured to identify genetic criteria defining the simulacrum, and to ensure that patients within the cohort meet the genetic criteria.

9. The system of claim 1 wherein:
the controller is further configured to deploy a Large Language Model (LLM) that identifies the phenotypes by classifying data in the request into a target medical concept, consulting a graph data structure that includes an entry for the target medical concept, and selecting phenotypes associated with additional medical concepts within a threshold distance of the target medical concept within the graph data structure.

10. A method for anticipating potential medical conditions for patients, the method comprising:
retrieving Electronic Health Record (EHR) data for each patient within a population;
partitioning the EHR data for each patient into discrete time periods;
for each time period for each patient:
assembling a sentence comprising a combination of medical concepts for the patient during the time period, resulting in conversion of EHR data for that patient into multiple sentences;
identifying a request to generate a simulacrum for a subject;
identifying phenotypes defining the simulacrum;
retrieving sentences for a cohort of patients within the population having the identified phenotypes;
identifying shared medical concepts between the patients based on the retrieved sentences;
updating the simulacrum to include the shared medical concepts; and
transmitting the simulacrum toward a source of the request in response to the request.

11. The method of claim 10 further comprising:
identifying temporal relationships between the shared medical concepts; and
updating the simulacrum to include the temporal relationships.

12. The method of claim 11 wherein:
the temporal relationships comprise a mean or median time between diagnosis of a first of the shared medical concepts and a second of the shared medical concepts.

13. The method of claim 11 wherein:
identifying temporal relationships between the shared medical concepts comprises:
calculating, for patients within the cohort, a temporal distance between sentences having a first of the shared medical concepts and sentences having a second of the shared medical concepts; and
determining a likelihood that the temporal distance is indicative of the first of the shared medical concepts and the second of the shared medical concepts having a non-random association with each other in time.

14. The method of claim 10 wherein:
identifying the shared medical concepts comprises:
reviewing candidate medical concepts reported within sentences for patients within the cohort;
for each of the candidate medical concepts: comparing a prevalence within the cohort to a prevalence within the population; and
identifying a candidate medical concept as a shared medical concept in response to determining that a prevalence of the candidate medical concept is greater in the cohort than in the population.

15. The method of claim 10 further comprising:
categorizing medical concepts based on expected impact to patient health; and
filtering contents of the simulacrum to exclude shared medical concepts having less than a threshold impact on patient health.

16. The method of claim 10 further comprising:
dynamically assembling the cohort for the patient by excluding patients having an age of presentation for at least one of the phenotypes which differs by more than a threshold amount from an age of presentation for the subject.

17. The method of claim 10 further comprising:
identifying genetic criteria defining the simulacrum; and
ensuring that patients within the cohort meet the genetic criteria.

18. The method of claim 10 further comprising:
deploying a Large Language Model (LLM) that identifies the phenotypes by:
classifying data in the request into a target medical concept;
consulting a graph data structure that includes an entry for the target medical concept; and
selecting phenotypes associated with additional medical concepts within a threshold distance of the target medical concept within the graph data structure.

19. A non-transitory computer readable medium embodying programmed instructions which, when executed by a processor, are operable for performing a method for anticipating medical conditions for patients, the method comprising:
retrieving Electronic Health Record (EHR) data for each patient within a population;
partitioning the EHR data for each patient into discrete time periods;
for each time period for each patient:
assembling a sentence comprising a combination of medical concepts for the patient during the time period, resulting in conversion of EHR data for that patient into multiple sentences;
identifying a request to generate a simulacrum for a subject;
identifying phenotypes defining the simulacrum;
retrieving sentences for a cohort of patients within the population having the identified phenotypes;
identifying shared medical concepts between the patients based on the retrieved sentences;
updating the simulacrum to include the shared medical concepts; and
transmitting the simulacrum toward a source of the request in response to the request.

20. The medium of claim 19 wherein the instructions are further operable for:
identifying temporal relationships between the shared medical concepts; and
updating the simulacrum to include the temporal relationships.
